(19) United States
(12) Patent Application Publication
(10) Pub. No.: US 2024/0398980 A1
(43) Pub. Date: Dec. 5, 2024

(54) NUCLEIC ACID-CONJUGATED POLYMERIC NANOPARTICLES AND METHODS OF USE

(71) Applicants: The Brigham and Women's Hospital, Inc., Boston, MA (US); MASSACHUSETTS INSTITUTE OF TECHNOLOGY, Cambridge, MA (US)

(72) Inventors: Natalie Artzi, Cambridge, MA (US); Pere Dosta Pons, Cambridge, MA (US); Alexander M Cryer, Cambridge, MA (US)

(21) Appl. No.: 18/562,616

(22) PCT Filed: May 19, 2022
(86) PCT No.: PCT/US2022/030078

§ 371 Date: Nov. 20, 2023

Related U.S. Application Data
(60) Provisional application No. 63/190,358, filed on May 19, 2021.

(51) Int. Cl. A61K 47/69; A61K 47/59; A61K 47/64; A61P 35/00; C12N 15/88

(52) U.S. Cl. CPC A61K 47/6935; A61K 47/593; A61K 47/6455; A61P 35/00; C12N 15/88

(57) ABSTRACT

A nanoparticle composition including: a first polymer conjugated to a drug by a linker to form a first polymer compound, the first polymer compound having a net negative charge; and a second polymer conjugated to at least one positively charged group to form a second polymer compound, the second polymer compound having a net positive charge, and the first polymer compound and the second polymer compound interacting electrostatically to form a nanoparticle.

### CROSS-REFERENCE TO RELATED APPLICATIONS

The present application claims priority to U.S. Provisional Patent Application No. 63/190,358 that was filed May 19, 2021, the entire contents of which are hereby incorporated by reference.

### FIELD OF THE INVENTION

The disclosed technology is generally directed to polymeric nanoparticles for drug delivery. More particularly the technology is directed to electrostatically complexed polymeric nanoparticles for drug delivery.

### BACKGROUND OF THE INVENTION

Biomaterial-based delivery strategies can be leveraged to improve internalization of drugs into cells, therefore augmenting their therapeutic efficacy. It has been previously demonstrated that using nanoparticles of lipidic or polymeric origin, including as liposomes, to encapsulate nucleic acids can improve cell internalization. However, liposomes suffer from poor packaging capacity and limited storage stability. Further, the encapsulation of small hydrophilic molecules within polymeric nanoparticles remains challenging.

### BRIEF SUMMARY OF THE INVENTION

Accordingly, disclosed herein are electrostatically complexed polymeric nanoparticles with improved stability, cytosolic localization, and endosomal escape, resulting in greater therapeutic response. Embodiments of the present disclosure provide an electrostatically complexed nanoparticle composition and methods for its use in enhanced therapeutics delivery.

The nanoparticle composition comprises a first polymer compound and a second polymer compound. The first polymer compound comprises a first polymer conjugated to a drug via a linker and has a net negative charge. The drug may be a nucleotide which, in one embodiment, is a cyclic dinucleotide (CDN) that can act as a stimulator of interferon genes (STING) agonist as a promising activator of antitumor immunity. In one embodiment the linker may be an enzyme-sensitive linker which is cleaved by cathepsin in the cell endosome and which facilitates lysosomal proteolysis of the nucleotide into its free form in the cell.

The second polymer compound comprises a polymer conjugated to at least one positively charged group and has a net positive charge. In one embodiment the positive functional group is arginine polypeptide.

The first net negatively charged polymer compound and the second net positively charged polymer compound interact electrostatically to form a nanoparticle. In one embodiment the first polymer compound comprises no more than 3.1% w/w of the final composition. In one embodiment the nanoparticle composition can be further modified to increase circulatory half-life of the nanoparticle composition by PEGylation.

In one embodiment, a pharmaceutical composition of the nanoparticles may be administered to a subject in need of treatment. In one embodiment the nanoparticle composition encounters a first cell, preferably a cancer cell. In one embodiment the nanoparticle composition remains in the first cell for no less than 24 hours. In one embodiment the nanoparticle composition is transferred from the first cell to second cell. In one embodiment the second cell is a macrophage or a dendritic cell. In this way, the nanoparticle composition's enables cancer cells to act as a reservoir of CDN and contributes to the notably potent innate and adaptive antitumor responses leading to largely curative outcomes, even after tumor rechallenge.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows a schematic, optimization, and characterization of conjugated CDN-NPs. Panel a) shows synthesis of cationic pBAEs and conjugated pBAEs formulations. Cationic pBAEs are formulated by mixing acrylate-terminate pBAE polymer with arginine oligopeptide (termed C6-CR3). Maleimide-modified ML-317 is conjugated to pBAE by Diels-Alder reaction (termed ML-317-Linker-pBAE). After CDN conjugation, ML-317-Linker-pBAE polymer is electrostatically complexed with C6-CR3 polymer, resulting in the formation of covalently conjugated CDN-NP. CDN-NPs are PEGylated using NHSPEG, purified and sterilized by filtration. CDN is released from the CDN-NPs though a cathepsin-cleavable linker in the cell cytoplasm. Panel b) shows the therapeutic efficacy of CDN-NPs was studied using different murine preclinical tumor models. We further investigated the role of cancer cells and host cells, particularly immune cells, in the context of CDN-NP therapeutic efficacy. Panel c) shows electrostatically complexed CDN to arginine-modified pBAE failed to activate IFN-I after several hours of incubation in PBS. Comparison of IRF response generated by fresh and 48 h incubated CDN polyplexes (n=3 biologically independent samples). Panel d) shows biophysical characterization of CDN-conjugated nanoparticles was determined by dynamic light scattering (DLS). Panel e) shows CDN-NP digestion using papain enzyme. CDNNPs were incubated with papain enzyme and total CDN released was quantified at different time points by LC-MS-MS. Panel f) shows CDN-NPs stability study in mouse plasma was determined at different time points by LC-MS-MS. Panel g) shows dose-response curves of the TRF response produced by CDN-NPs or free CDN in THP-1 Dual™ cell line (n=4 biologically independent samples). Panel h) shows dose-response curves of the NFkB response produced by CDN-NPs or free CDN in THP-1 Dual™ cell line (n=4 biologically independent samples). Panel i) shows dose-response curves of the IRF response produced by CDN-NPs or free CDN in RAW 264.7 reporter cell line (n=4 biologically independent samples). Panel j) shows a comparison of IRF response generated by fresh and 48 h incubated CDN-NPs in PBS (n=3 biologically independent samples).

FIG. 2: Panel a) shows the chemical structure of C6 polymer. Panel b) shows the chemical structure of C32 polymer. Panel c) shows the chemical structure of C6-CR3 polymer. Panel d) shows the chemical structure of C6-CK3 polymer. Panel e) shows the chemical structure of C6-CH3 polymer. Panel f) shows the chemical structure of C32-Furan polymer.

FIG. 3 shows synthesis, optimization, and characterization of electrostatic CDN-pBAE polyplexes. Fluorescent and non-fluorescent 2,3-cGAMP was used as CDN for the electrostatic pBAE NP optimization. Panel a) shows a scheme of oligopeptide-modified pBAE complexation with CDN molecules. Panel b) shows an agarose retardation assay of arginine modified pBAE using fluorescent CDN (CDN-F). Polyplexes were formed using CDN-F and arginine-modified pBAE at indicated w/w ratios and loaded onto an agarose gel to assess CDN-F mobility by electrophoresis. Panel c) shows the size, polydispersity, and Z-potential of complexed CDN using different oligopeptide-end modified pBAEs (determined by DLS). Panel d) shows the effect of oligopeptide-end modified pBAEs on CDN polyplex stability in PBS (determined by DLS). Panel e) shows dose-response curves of the IFN-I response produced by CDN-containing nanoparticles or free CDN in THP-1 reporter cell line (n=4 biologically independent samples). Panel f) shows dose-response curves of the NF-kB response produced by CDN-containing nanoparticles or free CDN in THP-1 reporter cell line (n=4 biologically independent samples).

FIG. 4 shows optimization and characterization of CDN-conjugated NPs. Panel a) shows the size, polydispersity, and Z-potential of pBAE-CDN NPs containing different amount of CDN (determined by Dynamic Light Scattering (DLS)). Panel b) shows a nanoparticle size distribution histogram determined by DLS. Panel c) shows the effect of CDN loading efficiency on polyplex stability in PBS (determined by DLS).

FIG. 5 shows optimization of PEGylated CDN-NPs. Panel a) shows the size, and Panel b) show the Z-potential of PEGylated and non-PEGylated CDN NPs (determined by DLS). Panel c) shows a comparison between the IRF response produced by PEGylated or non-PEGylated CDN NPs in THP-1 reporter cell line (n=3 biologically independent samples).

FIG. 6 shows representative histogram images of CDN quantification from pBAE NPs using LCMS-MS. Panels a) and b) show CDN-NPs before (a) and after (b) cathepsin cleavage using papain enzyme.

FIG. 7 shows cell viability profiles of different concentrations of electrostatic and conjugated CDN-pBAE NPs was analyzed 24 h post treatment. Samples were normalized to untreated cells. Data are represented as mean±SD (n=3).

FIG. 8 shows dose-response curves of the IFN-I and NF-κB response produced by CDN-NP, NP-control and free CDN in THP-1 STING KO reporter cell line (n=4 biologically independent samples). Panel a) shows the dose-response curve of the TRF fold change and panel b) shows the NF-κB fold change.

FIG. 9 shows flow cytometric quantification of the uptake of CDN-NPs in THP-1 Dual™ and RAW 264.7 cells (n=3 biologically independent samples).

FIG. 10 shows how CDN-NPs enhance the immunostimulatory activity of CDN. Panel a) shows a representative flow cytometry plot of MHCII and CD86 expression in BMDCs. Panel b) shows flow cytometric quantification of CD86 expression in dendritic cells (DCs; CD86+CD11c+MHC-II+) 24 h following CDN-NP treatment at different CDN doses. Data are presented as mean±}s.e.m. (n=3). Panel c) shows flow cytometric quantification of CD80 expression in dendritic cells (DCs; CD80+CD11c+MHC-II+) 24 h following CDN-NP treatment at different CDN doses. Data are presented as mean±}s.e.m. (n=3). Panel d shows flow cytometric quantification of activated dendritic cells (DCs; MHC-IIhi CD11c) 24 h following CDN-NP treatment at different CDN doses. Data are presented as mean±}s.e.m. (n=3). Panels e-f) show representative flow cytometry histogram (left) and quantification (right) of CD86 expression by macrophages (MΦ; CD11b+F4/80+) following CDN-NP treatment. Data are presented as mean±}s.e.m. (n=3). Panel g) shows flow cytometric quantification of CD80 expression in macrophages (MΦ; CD80+CD11b+F4/80+) 24 h following CDN-NP treatment at different CDN doses. Data are presented as mean±}s.e.m. (n=3). Panel h shows M1/M2 ratio 24 h following CDN-NP treatment at different CDN doses. Inflammatory M1-like was defined as CD86 expression and anti-inflammatory M2-like was defined as CD206 expression. Data are presented as mean±}s.e.m. (n=3). Panel i) shows the dose response of the CDN-NP cell internalization in B16 F10, 4T1, CT26, BMDCs and BMDMs. Panel j) shows a scheme of cancer cells acting as a reservoir of CDN-NP, which are transferred to DCs or macrophages. Panel k) shows the percentage of immune cells containing CDN-NP transferred from cancer cells. Panels 1-m) show IRF3 response produced by transferred CDN-containing nanoparticles or free CDN from B16 wt or B16−/− to (k) THP-1wt or THP-1−/− and (1) Raw^{wt} or Raw^{-/-}. Data are presented as mean±}s.e.m. (n=3). n-o, CD86 expression by transferred CDN-containing nanoparticles or free CDN from B16 wt or B16−/− to (m) DCs^{wt} or DCs^{-/-} and (n) MΦ or MΦ^{-/-}. Data are presented as mean±}s.e.m. (n=3). In panels b, c, d, f, g, h, k, 1, m, n and o data were reported by Mean Fluorescence Intensity (MFI) normalized to untreated cells and the statistical significance was determined between CDN, Empty-NP and CDN-NP conditions by ordinary two-way ANOVA with Tukey's multiple comparison test. ***P≤0.001, **P≤0.01, *P≤0.05.

FIG. 11 shows CDN-NP distribution, cell internalization, and cytokine activation after i.v. administration. Panel a) shows a schematic timeline for PK and BD studies following CDN-NP systemic delivery in mice bearing B16 tumors. Panel b) shows pharmacokinetic analysis of fluorescent CDN-NPs upon i.v. administration (n=3-4 biologically independent samples). Panel c) shows representative IVIS images of organs. Distribution of total adjusted radiant efficiency within each time point in the spleen, kidneys, liver, lungs, heart, tdLN, and tumor. Panel d) shows fluorescent CDN-NPs quantification in organs (n=3-4 biologically independent samples). Panels e-h) show cellular internalization of fluorescent CDN-NPs in different tissues; tdLN (e), tumor (f), spleen (g), and liver (h). Data are presented as mean±s.e.m. (n=5 samples per group). Statistical significance was determined between 4 h and 24 h time points by ordinary two-way ANOVA with uncorrected Fisher's LSD multiple comparison test. ***P≤0.001, **P≤0.01, *P≤0.05. Panel i) shows cytokine profile 4 h after systemic administration of CDN-NP, NP control, or free CDN in the tumor, spleen, tdLN and plasma. Values are represented as log 10 fold over untreated animals (n=5 samples per group).

FIG. 12 shows CDN-NP improve therapeutic outcome of CDN and synergize with ICB. Mice with 30-50 mm³ subcutaneous tumors were administered CDN-NPs, free CDN, or PBS intravenously (i.v.) three times, 4 days apart. CDN therapy was combined with and without ICB, aPD-1 mAb, administered intraperitoneally (i.t) 24 h post CDN i.v. delivery, three times. Panel a) shows B16 F10 melanoma bearing mice tumor growth. (n=10-12, data are means±s.e.m.). Panels b and c) show individual tumor growth curves (b) and Kaplan-Meier survival curves (c) of B16-F10 mice treated with different therapeutic combinations (n=10-12 biologically independent samples). Panel d) shows CT-26 colon tumor-bearing mice growth. (n=8-13, data are means±s.e.m.). Panels e and f) show individual tumor growth curves (e) and Kaplan-Meier survival curves (f) of CT-26 tumor-bearing mice treated with different therapeutic combinations (n=8-13 biologically independent samples). Panel g) shows 4T1 triple negative breast tumor-bearing mice growth. (n=8-9, data are means±s.e.m.). Panels h and i) show individual tumor growth curves (h) and Kaplan-Meier survival curves (i) of 4T1 tumor-bearing mice treated with different therapeutic combinations (n=8-9 biologically independent samples). Panel j) shows a rechallenge scheme of cured B16-F10 and CT-26 tumor-bearing mice. Cured mice were injected with 5×10⁵ B16-F10 or 1×10⁵ CT-26 cells in the opposite side of the flank at day 60 post first treatment. Tumor growth and survival was monitored. Panel k) shows Kaplan-Meier survival curves of B16-F10 tumor-bearing mice. Panel 1) shows Kaplan-Meier survival curves of CT-26 tumor-bearing mice. In panels a, d, and g, the statistical significance was determined by Kruskal-Wallis with Dunn's multiple comparisons test. ****P≤0.0001, ***P<0.001, **P≤0.01, *P≤0.05. In panels c, f, i, k, and 1, the statistical significance was determined against the untreated group unless indicated otherwise by Mantel-Cox test. ****P≤0.0001, ***P≤0.001, **P≤0.01, *P≤0.05.

FIG. 13 shows how CDN-NPs activate innate and adaptive anti-tumor immune responses. B16F10 tumors, tdLN, and spleen were collected from mice 2 days and/or 7 days after treatment. Panel a) shows representative flow cytometry histogram (left) and quantification (right) of activated dendritic cells (CD86hi CD11c+MHCII+CD45+) 48 h following intravenous injection in the TME, tdLN, and spleen. Data are presented as mean±s.e.m. (n=5). Panel b) shows representative flow quantification of activated resident DCs (CD86hi CD8+CD11c+MHCII+CD45+) 48 h following intravenous injection in the TME, tdLN, and spleen. Data are presented as mean±s.e.m. (n=5). Panel c) shows relative quantification of M1-like macrophages (CD86hi) and M2-like macrophages (CD206hi) ratio gating on F4/80+CD11b+CD45+ cells. Data are presented as mean±s.e.m. (n=5). Panels d and e) show NK cell proliferation (ki67+NK1.1+) (d) and activation (CD69+NK1.1+) (e) was quantified by flow cytometry 7d following intravenous injection in the TME. Data are presented as mean±s.e.m. (n=5). Panel f) shows Ratio of CD8+ to CD4+ T cells in the TME at 2d and 7d following treatment. Data are presented as mean±s.e.m. (n=5). Panels g, h, and i) show representative flow cytometric analysis images (g), and the relative quantification of IFNg+CD4+(h) and IFNg+CD8+(i) 7d following intravenous injection in the TME. Data are presented as mean s.e.m. (n=5). Panel j) shows CD4 cell proliferation (ki67+CD4+CD3+) was quantified by flow cytometry 7d following intravenous injection in the tdLN. Panels k and 1) show central CD4+ cell memory (CD44+CD62L+) (k) and effector CD4+ cell memory (CD44+CD62L-) (1) in the tdLN 7d following treatment. Data are presented as mean±s.e.m. (n=5). Panel m) shows CD8 cell proliferation (ki67+CD8+CD3+) was quantified by flow cytometry 7d following intravenous injection in the tdLN. Data are presented as mean±s.e.m. (n=5). Panel n and o) show central CD8+ cell memory (CD44+CD62L+) (n) and effector CD8+ cell memory (CD44+CD62L−) (o) in the tdLN 7d following treatment. Data are presented as mean±s.e.m. (n=5). In panels a, b, and c the statistical significance was determined between untreated, aPD-1, CDN, CDN-NP, CDN+aPD-1 and CDN-NP+aPD-1 conditions by ordinary two-way ANOVA with Tukey's multiple comparison test. ****P≤0.0001, ***P≤0.001, **P≤0.01, *P≤0.05. In panels d, e, f, h, i, j, k, 1, m, n and o, the statistical significance was determined between untreated, aPD-1, CDN, CDN-NP, CDN+aPD-1 and CDN-NP+aPD-1 conditions by Kruskal-Wallis with uncorrected Dunn's multiple comparisons test. ****P≤0.0001, ***P≤0.001, **P≤0.01, *P≤0.05.

FIG. 14 shows that STING activation within host cells is sufficient to promote anti-tumor immunity. Panel a) shows treatment scheme for the confirmation of CDN Sting-dependent efficacy in the B16-F10 melanoma model. B16-F10 STING^{-/-} or B16-F10 while type (WT) tumor cells were implanted in C57BL/6 Sting^{-/-} or C57BL6 while type (WT) mice following CDN-NP treatment when the tumor size reached 30-50 mm³. Panel b) shows average tumor-growth kinetics after treatment (n=8 data are means±s.e.m.). Panel c) shows Kaplan-Meier survival curves (n=8 biologically independent samples). Panel d) shows individual tumor growth curves. Panel e and f) show mice were subjected to splenectomy one week before tumor induction. Then, mice with 30-50 mm³ subcutaneous tumors were treated with CDN-NPs with and without aPD-1mAb or PBS. CDN therapy was administrated intravenously (i.v.) three times, 4 days apart. aPD-1 mAb therapy was administrated intraperitoneally (i.t) 24 h post CDN treatment. (e) B16-F10 melanoma-bearing mice tumor growth (n=8, data are presented as means±s.e.m.). (f) Kaplan-Meier survival curves. Panels g), h), and j) show tumor growth and survival using CDN-NP or NP Control treated (g,h) B16-F10 wild type (B16^{wt}) and (i,j) B16-F10 STING KO (B16^{-/-}) cells. (n=5 data are presented as means±s.e.m.). In panels b) and e), the statistical significance was determined by Kruskal-Wallis with Dunn's multiple comparisons test. ****P≤0.0001, ***P≤0.001, **P≤0.01, *P≤0.05. In panels d), f), i), and k), the statistical significance was determined against the untreated group unless indicated otherwise by Mantel-Cox test. ****P≤0.0001, ***P≤0.001, **P≤0.01, *P≤0.05. In panels h) and j), the statistical significance was determined bymixed-effects model with uncorrected Fisher's LSD. ****P≤0.0001, ***P≤0.001, **P≤0.01, *P≤0.05.

FIG. 15 shows a maximum tolerated dose (MTD) using CDN-NPs. Mice with 30-50 mm³ subcutaneous tumors were administered using different concentrations of CDN-NPs intravenously (i.v.) three times, 4 days apart (n=3 animals).

FIG. 16 presents a CDN-NP biodistribution study. Panels a) and b) show organ CDN-NPs quantification using plate reader (a) and total adjusted radiant efficiency (b) within each time point in the spleen, kidneys, liver, lungs, heart, tdLN, and tumor. (n=3-4 biologically independent samples).

FIG. 17 shows that empty NPs did not affect tumor growth and overall survival. Mice with 30-50 mm³ subcutaneous tumors were administered with empty NPs or PBS (untreated) intravenously (i.v.) three times, 4 days apart. Panel a) shows B16-F10 melanoma-bearing mice tumor growth. (n=5, data are means±SEM, Kruskal-Wallis test). Panel b) shows and Kaplan-Meier survival curves (n=5 biologically independent samples).

FIG. 18 shows mice body weight following CDN therapy. Mice with 30-50 mm³ subcutaneous tumors were administered CDN-NPs, free CDN, or PBS intravenously (i.v.) three times, 4 days apart. CDN therapy was combined with and without ICB, anti-PD-1, administered intraperitoneally (i.t) 24 h post CDN i.v. delivery, three times. Panel a) shows B16-F10 melanoma model. Panel b) shows CT-26 colon model. Panel c) shows 4T1 breast cancer model.

FIG. 19 shows that systemic delivery of CDN-NP stimulates proinflammatory cytokines and chemokines. Panel a) shows tumor; panel b) shows spleen; panel c) shows tdLN; and panel d) shows plasma samples were analyzed 4 h after CDN-NP, NPc control or free CDN systemic administration. Data shown as mean±s.e.m. (n=5).

FIG. 20 shows that reduced splenomegaly is observed in CDN-NP treated animals. Spleen weight of mice treated with the indicated formulations were measured 7 days post single administration. Data are represented as mean±s.e.m. (n=5 mice per group). Statistical significance was determined by Kruskal-Wallace test without Dunn's multiple comparison test. ****P≤0.0001, ***P≤0.001, **P≤0.01, *P£0.05.

FIG. 21 shows that pBAE-CDN NPs increase Dendritic cell population in the tdLN. Panel a) shows spleen, panel b) shows tumor, and panel c) shows tdLN. Representative flow quantification of DCs (CD11c+MHCII+CD45+) 48 h following intravenous injection in the TME, tdLN, and Spleen. Data are presented as mean±s.e.m. (n=5 mice per group). Statistical significance was determined by Kruskal-Wallace test without Dunn's multiple comparison test. ****P≤0.0001, ***P≤0.001, **P≤0.01, *P≤0.05.

FIG. 22 shows that CDN NPs activate migratory Dendritic Cells. Representative flow quantification of CD86⁺ migratory DCs (CD103+CD11c+MHCII+CD45+) 48 h following intravenous injection in the TME, tdLN, and Spleen. Data are presented as mean±s.e.m. (n=5-6 mice per group). Statistical significance was determined by ordinary two-way ANOVA with Tukey's multiple comparison test. ****P≤0.0001, ***P≤0.001, **P≤0.01, *P≤0.05.

FIG. 23 shows that CDN NPs increase plasmacytoid DCs in the Spleen and tumor. Representative flow quantification of plasmacytoid DCs (siglecH^{hi}CD11c⁺MHCII⁺CD45⁺) 48 h following intravenous injection in the TME, tdLN, and Spleen. Data are presented as mean±s.e.m. (n=5-6 mice per group). Statistical significance was determined by ordinary two-way ANOVA with Tukey's multiple comparison test. ****P≤0.0001, ***P≤0.001, **P≤0.01, *P≤0.05.

FIG. 24 shows that CDN-NPs promote an influx of immunosuppressive gMDSC. Panels a-b) show flow cytometric quantification of the number of monocytic (mMDSC;CD11b⁺Ly6⁺c⁺Ly6g⁻) Panel (a) shows and granulocytic (gMDSC; CD11b⁺Ly6c⁺Ly6g^{hi}) MDSCs and panel (b) shows 48 h following intravenous injection in the TME, tdLN, and Spleen. Data are presented as mean±s.e.m. (n=5-6 mice per group). Statistical significance was determined by ordinary two-way ANOVA with Tukey's multiple comparison test. ****P≤0.0001, ***P≤0.001, **P≤0.01, *P≤0.05.

FIG. 25 shows that CDN-NPs increase NK population in the spleen, TME, and tdLN. Representative flow quantification of NK (NK1.1⁺CD45⁺) 7 days following intravenous injection in the TME, tdLN, and Spleen. Data are presented as mean±s.e.m. (n=55-6 mice per group). Statistical significance was determined by ordinary two-way ANOVA with Tukey's multiple comparison test. ****P≤0.0001, ***P≤0.001, **P≤0.01, *P≤0.05.

FIG. 26 shows relative quantification of IFNγ⁺CD4⁺ and IFNγ⁺CD8⁺ 7d following intravenous injection in the tdLN. Data are presented as mean±s.e.m. (n=5-6 mice per group). Statistical significance was determined by Kruskal-Wallace test without Dunn's multiple comparison test. ****P≤0.0001, ***P≤0.001, **P≤0.01, *P≤0.05.

FIG. 27 shows that CDN NPs activate CD4 and CD8 T cell population in the tdLN. Panel a-b) shows CD4 cell activation (CD69⁺CD4⁺CD3⁺) (a) and CD8 cell activation (CD69⁺CD8⁺CD3⁺) (b) was quantified by flow cytometry 7d following intravenous injection in the tdLN. Data are presented as mean s.e.m. (n=5-6 mice per group). Statistical significance was determined by Kruskal-Wallace test without Dunn's multiple comparison test. ****P≤0.0001, ***P≤0.001, **P≤0.01, *P≤0.05.

FIG. 28 shows the flow cytometry T cell gating strategy.

FIG. 29 shows the flow cytometry T cell memory gating strategy.

FIG. 30 shows that CDN and CDN-linker shows higher IRF and NF-kB activation than 2.3cGAMP. Panels a) and b) show dose-response curves of the (a) TRF and (b) NF-kB response produced by different types of CDNs in THP-1 reporter cell line (n=3 biologically independent samples).

### DETAILED DESCRIPTION OF THE INVENTION

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

Disclosed herein is a therapeutic nanoparticle composition for enhanced therapeutic efficacy of nucleotides. The nanoparticles include a first polymer compound and a second polymer compound. The first polymer compound includes a first polymer conjugated to a drug (e.g. a nucleotide) via a linker (e.g. an enzyme-sensitive linker) and may have a net negative charge. The second polymer compound includes a polymer conjugated (e.g. covalently linked) to at least one positively charged group and may have a net positive charge. The first net negatively charged polymer compound and the second net positively charged polymer compound interact electrostatically to form a nanoparticle. In some embodiments, the nanoparticles may include only positively charged polymers conjugated to a drug. In one embodiment, a pharmaceutical composition of the nanoparticles may be administered to a subject in need of treatment.

In various embodiments, the drug may include compounds such as nucleotides or polynucleotides, small molecules (e.g. various pharmaceutical compounds), or polypeptides such as proteins. In certain embodiments the drug may be a pro-drug compound which is metabolized (e.g. cleaved by an enzyme) or otherwise activated inside a body of a subject. In one embodiment, the nucleotide includes a nucleotide with therapeutic properties. In one embodiment, the nucleotide includes a cyclic dinucleotide (CDN). In one preferred embodiment, the CDN includes a stimulator of interferon genes (STING) agonist, including cGAMP. In a preferred embodiment, the STING agonist is ML-317. In other embodiments, the STING agonist comprises at least one of 2,3-cGMAP, ADU-S100, cyclic-di-GMP, cyclic-di-AMP, 2′5′-cGAMP, 3′3′-GAMP, 2′3′-(G(s)A(s)MP, or DMXAA. The terms “polynucleotides” and “nucleic acid” as used herein refers to a polymer containing at least two deoxyribonucleotides or ribonucleotides in either single- or double-stranded form and includes DNA, RNA, and hybrids thereof, including Cas9 mRNA or other mRNAs whose protein products are directly involved in gene editing. DNA may be in the form of antisense molecules, plasmid DNA, cDNA, PCR products, or vectors. RNA may be in the form of small hairpin RNA (shRNA), messenger RNA (mRNA), antisense RNA, siRNA, miRNA, anti-miRNA, micRNA, multivalent RNA, dicer substrate RNA or viral RNA (vRNA), and combinations thereof.

The first polymer compound includes a first polymer conjugated to a nucleotide via an enzyme-sensitive linker. In one embodiment, the first polymer is a poly(β-amino ester) (pBAE). The molecular weight of pBAE may vary from 500 Da to 20,000 Da or more. The pBAE polymers disclosed herein are formed by the addition reaction of primary amines (5-amino-1-pentanol and hexylamine) to an excess of diacrylate (1,4-butanediol diacrylate), resulting in an acrylate-terminated polymer. Nevertheless, in various embodiments other primary amines and acrylates having different molecular weights could be used instead of, or in addition to, the particular compounds disclosed herein. One feature of the disclosed polymers is the modification of the acrylate-terminated polymer with methyl-3-furanthiol to facilitate covalent conjugation of nucleic acids or other groups and accordingly the present disclosure encompasses embodiments of the first polymer which facilitate conjugation of a compound (e.g. a drug) via the Diels-Alder reaction; other possible reaction chemistries are disclosed below. In other embodiments, the first polymer may include instead of, or in addition to, pBAE, at least one of dendrimers, PEI polymers, HPMA, cationic dextran, cationic chitosan, gelatin, or various types of polysaccharides. In general, the first polymer is selected for a property of being capable of covalently conjugating nucleotide compounds (e.g. STING agonists) while the second polymer is selected based on having a net positive charge.

The enzyme-sensitive linker is positioned between the nucleotide and the first polymer. In one embodiment, the enzyme-sensitive linker is cleavable by lysosomal enzymes such as cathepsin and thereby facilitates lysosomal proteolysis of the nucleotide into its free form within the cell. In various embodiments, the first polymer may be conjugated to the nucleotide by a linker that is sensitive to other parameters that can lead to drug release inside of cells, including parameters such as increased or decreased pH levels, redox reactions, or addition/removal of phosphate groups by kinases/phosphatases.

In one embodiment the nucleotide is first covalently bound to the linker followed by a subsequent conjugation of the linker-nucleotide molecule to the first polymer, which is also accomplished through a covalent bond. In some embodiments the conjugation of the first polymer to the nucleotide is via the Diels-Alder reaction between a maleimide functional group on the nucleotide and a furan functional group on one or more terminal ends of the first polymer. In various embodiments, the conjugation of the first polymer to the nucleotide may occur using a chemistry which involves at least one of maleimide, dibenzocyclooctyne, N-hydroxysuccinimide (NHS), thiol, azide, 2 2′-dipyridyl disulfide, amine, carboxylic acid, aldehyde, alcohol epoxide, acrylate, alkyne, or aziridine. In other preferred embodiments, two nucleotides are conjugated to the first polymer (e.g. one nucleotide being conjugated to each end of the polymer) to achieve high encapsulation efficiency. In still other embodiments, other cationic polymers may be used instead of, or in addition to, pBAE, including branched polymers such as dendrimers or polysaccharides such as chitosan or dextran, which could greatly increase the number of nucleotides linked per polymer from 2 as with pBAE to 50 or more.

The second polymer compound includes a polymer covalently bound to one or more positively charged functional groups. In one embodiment the positively charged functional group is an amino acid. In another embodiment, the preferred amino acid is arginine. In one preferred embodiment, both terminal ends of the second polymer are functionalized with one or more positively charged functional groups. In various embodiments, other amino acids that could be used instead of, or in addition to, arginine, include at least one of lysine, histidine, or arginine. In still other embodiments, the second polymer may include any type of small molecule that contains primary, secondary, or ternary amine groups.

Without being limited as to theory, in one embodiment the nanoparticle composition self assembles through the electrostatic interaction of the positively charged group of the second polymer compound and the net negatively charge of the first polymer compound. In one embodiment the nanoparticle composition contains no more than 3.1% w/w of the first polymer compound with the balance being made up of the second polymer compound; in other embodiments the nanoparticle composition may include up to 30% or more by weight of the first polymer compound, depending on the particular formulation of the first and second polymer compounds, with the remaining portion being made up of the second polymer compound. In one embodiment the nanoparticles have a diameter no smaller than 5 nm and no larger than 400 nm. In one preferred embodiment, the nanoparticles have a diameter no smaller than 20 nanometers and no larger than 50 nanometers. In one embodiment the Zeta potential surface charge of the nanoparticles is about 20 mV.

In another embodiment the nanoparticles are further treated to increase their circulatory half-life. In one embodiment, the nanoparticles are PEGylated to increase their circulatory half-life. In this embodiment, the Zeta potential surface charge of the nanoparticles decreases compared to the non-PEGylated nanoparticle composition. In one embodiment, the surface charge of the PEGylated nanoparticle composition is about 6 mV. In other embodiments, the nanoparticles may be treated with N-(2-Hydroxypropyl) methacrylamide (pHPMA), which can be used as a carrier to enhance therapeutic efficacy and limit side effects.

In one embodiment, the nanoparticle composition contacts a first cell. In one embodiment the first cell is a cancer cell. In another embodiment, the nanoparticle composition remains in the cancer cell for no less than 24 hours. In another embodiment, the nanoparticle composition transfers from the first cell to a second cell. In one preferred embodiment the second cell is a dendritic cell or macrophage. In various embodiments, the nucleotide is released from the NPs and activates one or more cellular pathways including the STING pathway, as disclosed herein.

### Pharmaceutical Compositions

The nanoparticle composition disclosed herein may be formulated as pharmaceutical compositions that include: an effective amount of one or more nanoparticle compositions and one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the nanoparticle composition at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the nanoparticle composition at the site of action is about 2 to 10 μM.

The nanoparticle composition utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended-release dosage form, pulsatile release dosage form, mixed immediate release and controlled release dosage form, or a combination thereof.

The nanoparticle composition utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The nanoparticle composition utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The nanoparticle composition utilized in the methods disclosed herein may be formulated as a pharmaceutical composition for delivery via any suitable route. For example, the pharmaceutical composition may be administered via oral, intravenous, intramuscular, intraarterial, subcutaneous, topical, aerosolization/inhalation, and/or pulmonary routes. Examples of pharmaceutical compositions for oral administration include capsules, syrups, concentrates, powders, and granules. The nanoparticle composition may also be administered by local delivery. “Local delivery,” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous, or intradermal injection. Local delivery does not preclude a systemic pharmacological effect. Furthermore, the nanoparticle composition may be administered as part of a combination therapy in which the nanoparticle composition is administered one or more of before, simultaneous with, or subsequent to administration of another therapy.

The nanoparticle composition utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions including the nanoparticle composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

The nanoparticle composition employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed nanoparticle composition, which effective amount is related to the daily dose of the compound to be administered. Each dosage unit may contain the daily dose of a given compound or each dosage unit may contain a fraction of the daily dose, such as one-half or one-third of the dose. The amount of each nanoparticle composition to be contained in each dosage unit can depend, in part, on the identity of the particular nanoparticle composition chosen for the therapy and other factors, such as the indication for which it is given. The pharmaceutical compositions disclosed herein may be formulated so as to provide quick, sustained, or delayed release of the active ingredient after administration to the patient by employing well known procedures. The nanoparticle composition for use according to the methods of disclosed herein may be administered as a single nanoparticle composition or a combination of nanoparticle compositions.

As indicated above, pharmaceutically acceptable salts of the nanoparticle composition are contemplated and also may be utilized in the disclosed methods. The term “pharmaceutically acceptable salt” as used herein, refers to salts of the nanoparticle composition which are substantially non-toxic to living organisms. Typical pharmaceutically acceptable salts include those salts prepared by reaction of the nanoparticle composition as disclosed herein with a pharmaceutically acceptable mineral or organic acid or an organic or inorganic base. Such salts are known as acid addition and base addition salts. It will be appreciated by the skilled reader that most or all of the nanoparticle compositions as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the nanoparticle compositions or salts, esters, and/or amides, thereof. Solvate forms may include ethanol solvates, hydrates, and the like.

Methods for Treating with the Nanoparticle Compositions

Methods for treating subjects with the nanoparticle compositions disclosed herein are provided. Suitably the method for treating a subject comprises administering to the subject an effective amount of one or more of the nanoparticle compositions disclosed herein or a pharmaceutical composition comprising the effective amount of one or more of the nanoparticle compositions disclosed herein. As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with one or more of the compounds disclosed herein. In some embodiments, the subject is responsive to therapy with one or more of the nanoparticle compositions disclosed herein in combination with one or more additional therapeutic agents. For example, a “subject in need of treatment” may include a subject in need of treatment for cancer. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

In some embodiments, the subject has a cancer and may show symptoms associated therewith. Symptoms associated with cancer can be varied depending on the location and severity of the disease. In some instances, the cancer is located in or on the skin or an inner organ, tissue, or fluids, such as breast, lungs, heart, blood, bone, joints, or gastrointestinal tract.

Methods for inhibiting growth or proliferation of or killing a cancer are also provided. In some embodiments, administration of any of the compounds disclosed herein to a subject or contacting a cancer with the compound provides for inhibiting growth or proliferation of or killing the cancer.

In some embodiments, the methods described herein are practiced in vivo. In other embodiments, the methods described herein are practiced in vitro or ex vivo.

As used herein the term “effective amount” refers to the amount or dose of the nanoparticle composition that provides the desired effect. In some embodiments, the effective amount is the amount or dose of the nanoparticle composition, upon single or multiple dose administration to the subject, which provides the desired effect in the subject under diagnosis or treatment. Suitably the desired effect may be inhibiting the growth or proliferation of or killing the cancer in the subject or reverse the progression or severity of resultant symptoms associated with the cancer.

An effective amount can be readily determined by those of skill in the art, including an attending diagnostician, by the use of known techniques and by observing results obtained under analogous circumstances. In determining the effective amount or dose of nanoparticle composition administered, a number of factors can be considered by the attending diagnostician, such as: the species of the subject; its size, age, and general health; the degree of involvement or the severity of the disease or disorder involved; the response of the individual subject; the particular nanoparticle composition administered; the mode of administration; the bioavailability characteristics of the preparation administered; the dose regimen selected; the use of concomitant medication; and other relevant circumstances.

### Nanoparticles for Delivery of Nucleotides

Stimulator of interferon genes (STING) agonist is a promising activator of antitumor immunity. However, the systemic delivery of STING agonist, based on cyclic dinucleotides (CDNs), yields limited anticancer activity owing to poor serum stability and cellular internalization. Here, we show, in multiple models of murine cancer, that the intravenous administration of sub-microgram doses of chemically-modified CDNs covalently bound to poly(β-amino ester) nanoparticles (CDN-NP) results in potent innate and adaptive antitumor immune responses. The combination of CDN-NPs and immune checkpoint blockade led to largely curative outcomes, even after tumor re-challenge, in mouse models of melanoma and colon cancer and to survival benefit in a preclinical murine breast cancer model. The NPs activated STING signaling in immune cells in tumor and lymphoid tissues. We demonstrate, in a mouse melanoma model, that cancer-cell STING signaling is not necessary for therapeutic responses, but that cancer cells can act as a nanoparticle reservoir, releasing CDN to proximal immune cells.

Despite prolific advances in cancer immunotherapy, such as immune checkpoint blockade (ICB), many patients do not achieve complete tumor remission. Insufficient antitumor responses have spurred efforts to invoke complementary elements of the immune system, including the stimulator of interferon genes (STING) pathway. Agonism of STING is known to enact a type I interferon (IFN-I) driven proinflammatory program including the stimulation of macrophages, natural killer (NK) cells and dendritic cells (DCs). Subsequent cross presentation of tumor antigens to T cells in the tumor draining lymph nodes (tdLNs), predominantly by DCs, primes T cells for antitumor effector functionality, which may further sensitize the tumor microenvironment (TME) to ICB. Small molecule mimetics of endogenous ligands of STING, such as the cyclic dinucleotide (CDN) 2′3′-cyclic guanosine monophosphate-adenosine monophosphate (cGMP-AMP or cGAMP), have been synthesized to improve potency and stability, but have demonstrated limited antitumor efficacy when administered (primarily) intratumorally (IT) in clinical trials. Non-CDN derivatives have also been explored, with candidates emerging from small molecule library screens that have exhibited STING-mediated antitumor activity in vivo after systemic administration. Although this route of administration is desirable, the biodistribution (BD) and pharmacokinetics (PK) of such molecules may not be optimal for systemic STING agonism, reflected by the relatively high doses required to achieve therapeutic effect. Systemic administration of STING agonists is not only of valuable therapeutic utility for a broad range of malignancies and infectious diseases but can also be used as a tool to study the mechanistic underpinnings of STING-mediated antitumor efficacy, other STING-dependent biological activities, and the impact of different delivery strategies (e.g. antibody-drug conjugates, exosomes, etc.). Due to the challenges associated with efficacious delivery of small molecule CDN, such as their hydrophilicity and rapid degradation, nanotechnology can be leveraged to develop potent formulations. Nanoparticles (NPs) of lipid or polymeric origin have been developed for systemic administration, however, these formulations relied on encapsulation or electrostatic complexation of endogenous CDNs such as cGAMP, which raises issues regarding stability and potency in vivo.

To address these challenges, we report the development of a biodegradable, highly potent STING agonist in conjugate nanoparticle formulation for systemic delivery of sub-microgram doses of STING agonists, which give rise to increase in potency of at least one order of magnitude compared to previous STING agonists formulations. To achieve this, we chemically modified the recently identified STING agonist ML-317 (termed CDN), to contain an enzyme-sensitive linker cleavable by cathepsin in the cell endosome and capped it with a reactive functional group. The CDN was then covalently conjugated to poly(β-amino ester) (pBAE) (termed ML-317-Linker-pBAE) and self-assembled with arginine-modified pBAE to yield the CDN-NP formulation. PBAEs have been extensively optimized to increase their biocompatibility, in vivo stability, and fast degradation kinetics once they are internalized by cells. These modifications permitted robust cytoplasmic delivery of STING agonists and subsequent STING activation of immune cells in lymphoid tissues and the TME, leading to a type I IFN-I mediated innate antitumor immune response. CDN-NPs, particularly in combination with ICB, induced robust tumor rejection in multiple syngeneic murine tumor models. We further investigated the mechanistic determinants of antitumor efficacy by examining the BD and PK of the CDN-NPs and by performing in-depth downstream immune cell profiling by flow cytometry. We confirmed that DCs and macrophages across different tissues, including the TME, efficiently internalized CDN-NPs after intravenous (i.v.) delivery. This efficient internalization resulted in STING activation in several immune cell types, which aligns with our findings that STING activation within host immune cells is sufficient to promote anti-tumor immunity, as this effect was abrogated in STING knock-out (goldenticket) mice. Finally, we provide fundamental insights into the intercommunication between cancer cells and immune cells in the context of STING agonism and NPs. Interestingly, CDNNPs were transferred from cancer cells to immune cells, inducing their STING-specific activation, which occurred regardless of the STING-status of the cancer cells (i.e., wild type STING or STING knock out). This implies that STING activation in cancer cells is redundant for the antitumor activity of CDN-NPs, and in fact cancer cells act as a reservoir for CDN-NPs. This CDN-conjugate NP reaffirms the therapeutic potential of systemic STING agonism and illustrates how NPs can be engineered to improve the pharmacological properties of CDNs which directly impacts with their antitumor efficacy.

### EXAMPLES

The following are non-limiting descriptions of exemplary procedures for making and using nucleotide-linked nanoparticles according to embodiments of the disclosure:

### Example 1: Synthesis and Characterization of pBAE Polymers

Materials: All reagents and solvents were purchased from Sigma Aldrich unless otherwise stated. Arginine peptide (H-Cys-Arg-Arg-Arg-NH2) was obtained from CPC Scientific with a purity of at least 90%. TCDN-2 and TCDN-2 Mal were provided by Takeda Pharmaceuticals.

Synthesis of pBAE polymers: Polymers were synthesized in accordance with previous work. By an addition reaction of primary amines to an excess of diacrylate, resulting in an acrylate-terminated polymer (termed C6 polymer). C6 polymerization was performed by mixing 5-amino-1-pentanol (0.426 g, 4.1 mmol), hexylamine (0.422 g, 4.1 mmol), and 1,4-butanediol diacrylate (2.0 g, 9.1 mmol) under magnetic stirring at 90° C. for 24 hours. C32 polymerization was performed by mixing 5-amino-1-pentanol (0.852 g, 8.2 mmol) and 1,4-butanediol diacrylate (2.0 g, 9.1 mmol) under magnetic stirring at 90° C. for 24 hours.

¹H-NMR of C6 Polymer (400 MHz, Chloroform-d, TMS) (ppm): δ=6,41 (d, CH2═CH—), 6.15 (d, CH2═CH—), 5.87 (d, CH2═CH—), 4.21 (br, CH2-O—C(═O)—CH═CH2), 4.11 (t, -CH2-CH2-O—), 3.64 (t, CH2-CH2-OH), 2.79 (br, -CH2-CH2-N—), 2.46 (br, —N-CH2-CH2-C(═O)—O), 1.83-1.60 (br, —O-CH2-CH2-CH2-CH2-0), 1.40-1.18 (br, —CH2-CH2-CH2-CH2-OH, N—(CH2)2-CH2-(CH2)2-OH), 0.90 (t, CH2-CH2-CH3).

¹H-NMR of C32 Polymer (400 MHz, DMSO-d6, TMS) (ppm): δ=6,39 (d, CH2═CH—), 6.14 (d, CH2═CH—), 5.86 (d, CH2═CH—), 4.21 (br, CH2-OC(═O)—CH═CH2), 4.12 (t, -CH2-CH2-O—), 3.64 (t, CH2-CH2-OH), 2.79 (br, -CH2-CH2-N—), 2.45 (br, —N-CH2-CH2-C(═O)—O), 1.73 (br, —O-CH2-CH2-CH2-CH2-0), 1.58 (br, —CH2-CH2-CH2-CH2-OH), 1.36 (br, N—(CH2)2-CH2-(CH2)2-OH).

Cationic pBAE Synthesis (pBAE-CR3): C6 polymer was end-capped with different thiolterminated arginine peptide (H-Cys-Arg-Arg-Arg-NH2) at a 1:2.1 molar ratio in dimethyl sulfoxide (DMSO) and stirred overnight and room temperature. The resulting polymers were collected by precipitation in a mixture of diethyl ether and acetone (7:3 v/v) and dried in vacuo.

¹H-NMR of C6-CR3 Polymer (400 MHz, Methanol-d4, TMS) (ppm): δ=4.41-4.33 (br, NH2-C(═O)—CH—NH—C(═O)—CH—NH—C(═O)—CH—NHC(═O)—CH—CH2-, 4.16 (t, CH2-CH2-0), 3.58 (t, CH2-CH2-OH), 3.25 (br, NH2-C(═NH)—NH—CH2-, OH—(CH2)4-CH2-N—), 3.04 (t, CH2-CH2-N—), 2.82 (dd, -CH2-S—CH2), 2.48 (br, —N-CH2-CH2-C(═O)—O), 1.90 (m, NH2-C(═NH)—NH—(CH2)2-CH2-CH—), 1.73 (br, —O-CH2-CH2-CH2-CH2-0), 1.69 (m, NH2-C(═NH)—NH—CH2-CH2-CH2-), 1.56 (br, -CH2-CH2-CH2-CH2-OH), 1.39 (br, —N—(CH2)2-CH2-(CH2)2-OH), 0.88 (t, CH2-CH2-CH3).

¹H-NMR of C6-CK3 Polymer (400 MHz, Methanol-d4, TMS) (ppm): δ=4.38-4.29 (br, NH2-(CH2)4-CH—), 4.13 (t, CH2-CH2-O—), 3.73 (br, NH2-CH-CH2-S—), 3.55 (t, CH2-CH2-OH), 2.94 (br, CH2-CH2-N—, NH2-CH2-(CH2)3-CH—), 2.81 (dd, -CH2-SCH2), 2.57 (br, —N-CH2-CH2-C(═O)—O), 1.85 (m, NH2-(CH2)3-CH2-CH—), 1.74 (br, —O-CH2-CH2-CH2-CH2-0), 1.68 (m, NH2-CH2-CH2-(CH2)2-CH—), 1.54 (br, —CH2-CH2-CH2-CH2-OH), 1.37 (br, N—(CH2)2-CH2-(CH2)2-OH), 0.88 (t, CH2-CH2-CH3).

¹H-NMR of C6-CH3 Polymer (400 MHz, Methanol-d4, TMS) (ppm): δ=8.0-7.0 (br —N(═CH)—NH—C(═CH)—), 4.61-4.36 (br, -CH2-CH—), 4.16 (t,CH2-CH2-O—), 3.55 (t, CH2-CH2-OH), 3.18 (t, CH2-CH2-N—), 3.06 (dd, -CH2-CH—), 2.88 (br, OH—(CH2)4-CH2-N—), 2.82 (dd, -CH2-S-CH2-), 2.72 (br, —N-CH2-CH2-C(═O)—O), 1.75 (br, —O-CH2-CH2-CH2-CH2-0), 1.65 (m, NH2-CH2-CH2-(CH2)2-CH—), 1.58 (br, -CH2-CH2-CH2-CH2-OH), 1.40 (br, N—(CH2)2-CH2-(CH2)2-OH), 0.88 (t, CH2-CH2-CH3).

Synthesis of pBAE-furan polymer: C32 polymer was end-capped with methyl-3-furanthiol at 1:2.5 molar ratio in tetrahydrofuran (TIF) and stirred overnight at room temperature. The resulting polymers were collected by precipitation in diethyl ether and dried in vacuo. ¹H-NMR of C32-Furan Polymer (400 MHz,Chloroform-d, TMS) (ppm): δ=6,39 (d, CH2═CH—), 6.14 (d, CH2═CH—), 5.86 (d, CH2═CH—), 4.21 (br,CH2—O—C(═O)—CH═CH2), 4.12 (t, -CH2-CH2-O—), 3.64 (t, CH2-CH2-OH), 2.79 (br, -CH2-CH2-N—), 2.45 (br, —N-CH2-CH2-C(═O)—O), 1.73 (br, —O-CH2-CH2-CH2-CH2-0), 1.58 (br, —CH2-CH2-CH2-CH2-OH), 1.36 (br, N—(CH2)2-CH2-(CH2)2-OH), 1.27 (s, CH3-C(—O)═C(—S)—CH═).

Synthesis of end-modified pBAE-Furan polymer: For CDN conjugation, pBAE-furan polymer was end-capped with maleimide-modified CDN at 1:2 molar ratio in DMSO and stirred overnight at room temperature. Fluorescent polymer was generated likewise by end-capping pBAE-furan polymer with Alexa Fluor™ 647 C2 Maleimide (Thermo Fisher).

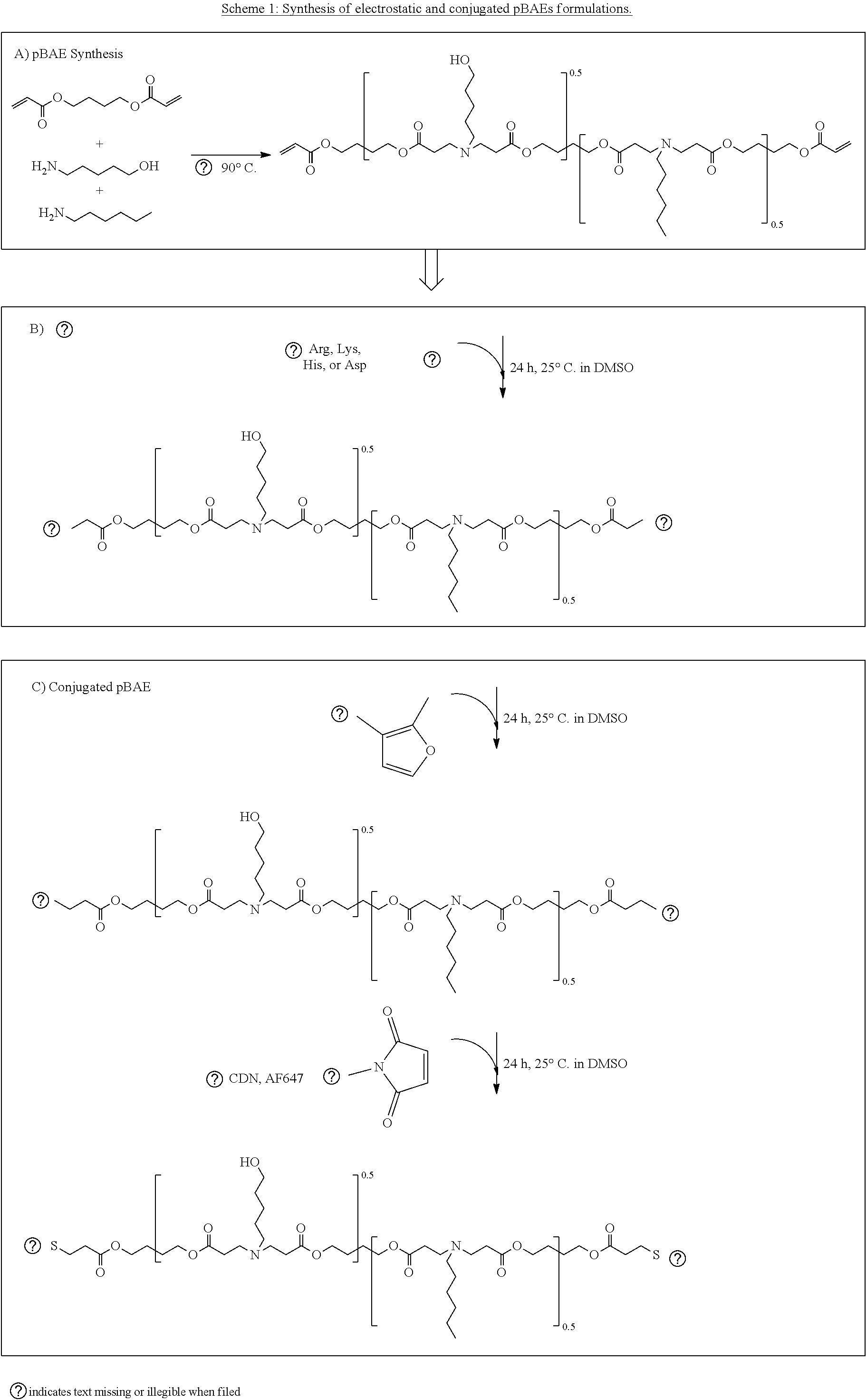

a, Synthesis of pBAE polymer. Combinations of 5-amino-1-pentanol, 1-hexylamine, and 1,4-butanediol diacrylate were used for the synthesis of pBAEs polymers. b, Cationic pBAEs are formulated mixing acrylate-terminate pBAE polymer with different polypeptide moieties. The R″ terminal can be an arginine-, lysine- and histidinepolypeptide. c, Conjugated pBAES are formulated by the addition of methyl-3-furanthiol to acrylateterminate pBAE polymer. Then, maleimide-modified CDN or maleimide-modified AF646 is conjugated to pBAE by Diels-Alder reaction.

Polymer characterization: Synthesized products were dissolved in an appropriate deuterated solvent and structures were confirmed by 1H-NMR, recorded in a 400 MHz Varian (NMR Instruments, Clarendon Hills, IL) (Supporting Information). Molecular weight (MW), relative to polystyrene standard, was determined by high performance liquid chromatography (HPLC Elite LaChrom system of VWRHitech equipped with a GPC Shodex KF-603 column and THE mobile phase).

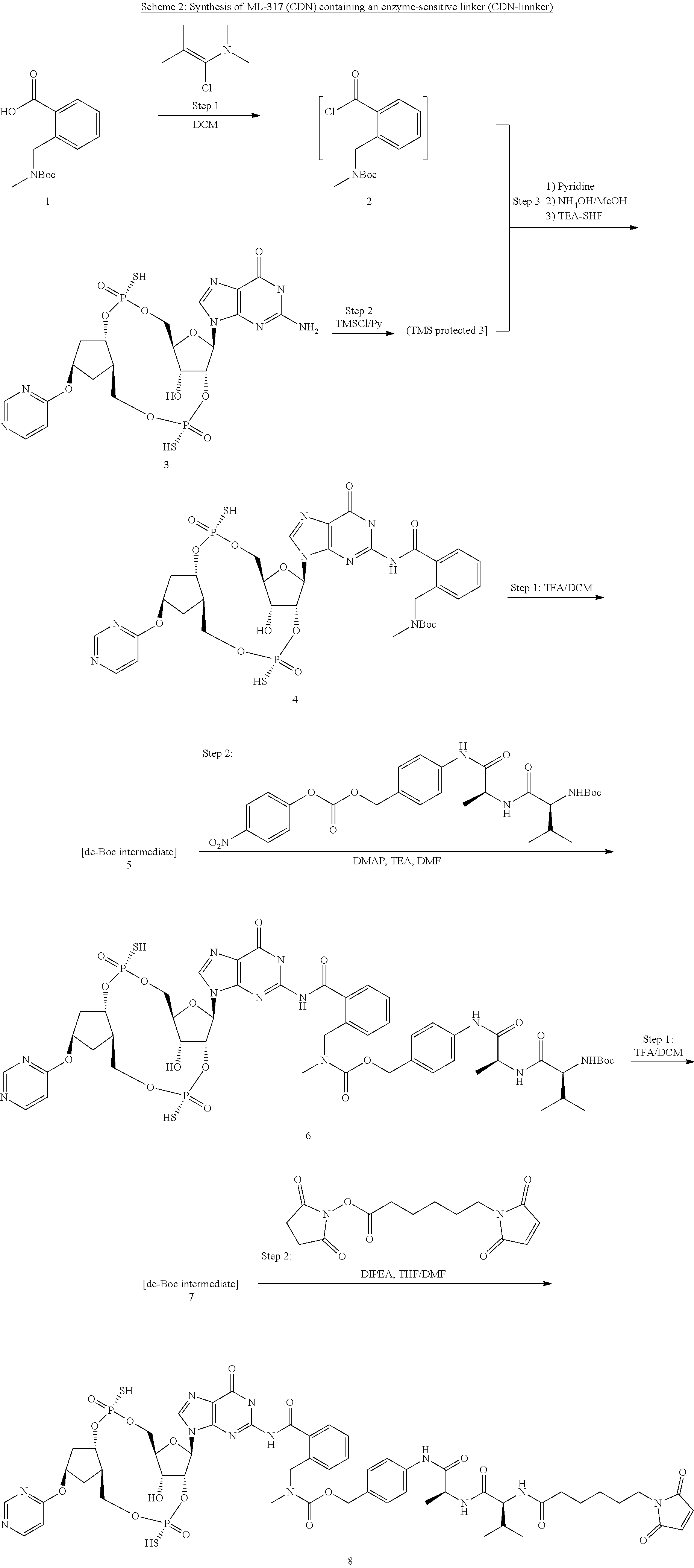

### Synthesis of CDN-Linker:

### A) Preparation of tert-Butyl [2-({9-[(2R,5R,7R,8R,10R,12aR,14R,15aS,16R)-16-hydroxy-2,10-dioxido-14-(pyrimidin-4-yloxy)-2,10-disulfanyldecahydro-5,8 methanocyclopenta[1][1,3,6,9,11,2,10]pentaoxadiphosphacyclotetradecin-7-yl]-6-oxo-6,9-dihydro-1H-purin-2-yl}carbamoyl)benzyl]methylcarbamate (4)

Step 1: 1-Chloro-N,N,2-trimethylpropenylamine (0.52 mL, 4.0 mmol, 2.0 equiv.) was added slowly to a solution of 2-(((tert-butoxycarbonyl)(methyl)amino)methyl)benzoic acid (525 mg, 2.0 mmol) in DCM (16 mL) at 0° C. The reaction mixture was then warmed to rt and stirred for 1 h. The reaction mixture was then concentrated to dryness to provide crude tert-butyl N-[(2-chlorocarbonylphenyl)methyl]-N-methyl-carbamate 2 (562 mg, 100%). The reaction was scaled as necessary for the amount needed for the next acylation step.

Step 2: 2-Amino-9-[(2R,5R,7R,8R,10R,12aR,14R,15aS,16R)-16-hydroxy-2,10-dioxido 14-(pyrimidin-4-yloxy)-2,10-disulfanyldecahydro-5,8 methanocyclopenta[1][1,3,6,9,11,2,10]pentaoxadiphosphacyclotetradecin-7-yl]-1,9-dihydro-6H-purin-6-one as the triethylamine salt (3 [see J. Med Chem. 2021, 64, 10, 6902-6923], 160 mg, 0.19 mmol) was dissolved in dry pyridine and concentrated to dryness (3×2 mL) and then placed under vacuum for 15 min. The residue was dissolved in pyridine (3 mL) under an argon atmosphere and chlorotrimethylsilane (0.15 mL, 1.13 mmol, 5.95 equiv.) was added. The reaction mixture was allowed to stir at rt for 30 min.

Step 3: Crude acid chloride 2 (800 mg, 2.82 mmol, 14.8 equiv.) from Step 1 dissolved in dry pyridine (3 mL) was added into reaction mixture of Step 2 via syringe. The reaction mixture was stirred at rt under an argon atmosphere for 16 h. The reaction mixture was then concentrated to dryness and MeOH (10 mL) and ammonium hydroxide (28-30% solution in water, 10 mL) were added and allowed to stir for 30 min. The reaction mixture was concentrated to dryness and the residue was dissolved in MeOH (15 mL). Triethylamine trihydrofluoride (0.12 mL, 0.75 mmol) was added and the reaction mixture was stirred at rt for another 30 min. The reaction mixture was then concentrated to dryness and the crude residue was adsorbed onto Celite and purified by reverse phase flash column chromatography (0-50% Acetonitrile (ACN)/aqueous triethylammonium acetate (10 mM)) to provide tert-butyl [2-({9-[(2R,5R,7R,8R,10R,12aR,14R,15aS,16R)-16-hydroxy-2,10-dioxido-14-(pyrimidin-4-yloxy)-2,10-disulfanyldecahydro-5,8-methanocyclopenta[1][1,3,6,9,11,2,10]pentaoxadiphosphacyclotetradecin-7-yl]-6-oxo-6,9-dihydro-1H-purin-2-yl}carbamoyl) benzyl]methylcarbamate (4) as the triethylamine salt (120 mg, 0.11 mmol, 58% from 3). Nuclear magnetic resonance (NMR) spectra were recorded in the solvent reported on a 400 MHz Bruker spectrometer using residual solvent peaks as the reference. LCMS (AA): m/z=897.3 (M+H).1H NMR (400 MHz, CD30D) δ 8.70 (s, 1H), 8.49 (s, 1H), 8.41 (d, J=6.0 Hz, 1H), 7.70 (d, J=7.5 Hz, 1H), 7.58-7.52 (m, 1H), 7.44-7.39 (m, 1H), 7.33 (d, J=7.8 Hz, 1H), 6.85 (d, J=6.0 Hz, 1H), 6.17 (d, J=8.3 Hz, 1H), 5.64-5.58 (m, 1H), 5.49-5.44 (m, 1H), 5.08-5.02 (m, 1H), 4.88-4.78 (m, 1H), 4.76-4.67 (m, 2H), 4.37-4.21 (m, 3H), 4.06-4.00 (m, 1H), 3.83-3.74 (m, 1H), 2.82 (s, 3H), 2.60-2.32 (m, 4H), 1.54-1.46 (m, 1H), 1.42 (s, 9H).31P NMR (162 MHz, CD30D) δ 55.19 (s, 1P), 53.20 (s, 1P).

### B) Preparation of 4-{[(2S)-2-({(2S)-2-[(tert-Butoxycarbonyl)amino]-3-methylbutanoyl}amino)propanoyl]amino}benzyl [2-({9[(2R,5R,7R,8R,10R,12aR,14R,15aS,16R)-16-hydroxy-2,10-dioxido-14-(pyrimidin-4-yloxy)-2,10-disulfanyldecahydro-5,8-methanocyclopenta[1][1,3,6,9,11,2,10]pentaoxadiphosphacyclotetradecin-7-yl]-6-oxo-6,9-dihydro-1H-purin-2-yl}carbamoyl) benzyl]methylcarbamate (6)

Step 1: Compound 4 (50 mg, 0.045 mmol) was added to a round bottom flask and cooled to 0° C. A solution of trifluoroacetic acid (0.24 mL, 3.2 mmol, 71 equiv.) and DCM (0.58 mL) was then added via syringe and the reaction mixture was stirred at 0° C. for 30 min. The reaction mixture was then concentrated to dryness and placed under vacuum for 2 h to provide intermediate 5 as the 2,2,2-trifluoroacetate salt (41 mg, 0.045 mmol, 100%). LCMS (AA): m/z=797.1 (M+H).

Step 2: To a solution of tert-butyloxycarbonyl-valyl-alanyl-(4-aminobenzyl)-(4-nitrophenyl)carbonate (58 mg, 0.10 mmol, 2.0 equiv.) and 4-dimethylaminopyridine (12 mg, 0.10 mmol, 2.0 equiv.) in DMF (0.38 mL) and triethylamine (0.055 mL, 0.40 mmol, 8.0 equiv.) was added a solution of intermediate 5 (45 mg, 0.05 mmol) in DMF (1.5 mL) at rt. The reaction mixture was allowed to stir at rt for 15 min. Celite was added and the mixture was concentrated to dryness. The crude residue absorbed onto Celite was purified by reverse phase flash column chromatography (0-40% ACN/aqueous triethylammonium acetate (10 mM)) to provide compound 6 as the triethylamine salt (10 mg, 0.0082 mmol, 18% from 4). Nuclear magnetic resonance (NMR) spectra were recorded in the solvent reported on a 400 MHz Bruker spectrometer using residual solvent peaks as the reference. LCMS (AA): m/z=1216.3 (M+H).1H NMR (400 MHz, CD3OD) δ 8.70 (s, 1H), 8.43 (brs, 1H), 8.40 (d, J=5.9 Hz, 1H), 7.70 (d, J=7.5 Hz, 1H), 7.56-7.46 (m, 3H), 7.42-7.38 (m, 1H), 7.32-7.19 (m, 3H), 6.84 (d, J=5.5 Hz, 1H), 6.15 (d, J=7.3 Hz, 1H), 5.62-5.56 (m, 1H), 5.54-5.48 (m, 1H), 5.08-5.02 (m, 1H), 5.04 (s, 2H), 4.88-4.83 (m, 1H), 4.81-4.76 (m, 2H), 4.53-4.47 (m, 1H), 4.37-4.22 (m, 3H), 4.08-4.03 (m, 1H), 3.92-3.89 (m, 1H), 3.82-3.74 (m, 1H), 2.88 (s, 3H), 2.58-2.30 (m, 4H), 2.10-2.03 (m, 1H), 1.54-1.47 (m, 1H), 1.46-1.44 (m, 3H), 1.44 (s, 9H), 0.98 (d, J=6.8 Hz, 3H), 0.93 (d, J=6.8 Hz, 3H). 31P NMR (162 MHz, CD30D) δ 55.01 (s, 1P), 53.03 (s, 1P).

### C) Preparation of 4-{[(2S)-2-{[(2S)-2-{[6-(2,5-Dioxo-2,5-dihydro-1H-pyrrol-1 yl)hexanoyl]amino}-3-methylbutanoyl]amino}propanoyl]amino}benzyl [2-({9[(2R,5R,7R,8R,10R,12aR,14R,15aS,16R)-16-hydroxy-2,10-dioxido-14-(pyrimidin-4-yloxy)-2,10-disulfanyldecahydro-5,8-methanocyclopenta[1][1,3,6,9,11,2,10]Pentaoxadiphosphacyclotetradecin-7-yl]-6-oxo-6,9-dihydro-1H-purin-2-yl}carbamoyl)benzyl]methylcarbamate (8, CDN-linker)

Step 1: The preparation was conducted using similar procedure for Intermediate 5 instead of starting from compound 4 (10 mg, 0.008 mmol). De-Boc intermediate 7 was thus obtained as TFA salt (8.9 mg, 0.008 mmol, 100%) which is used directly for the next step. LCMS (AA): m/z=1116.3 (M+H).

Step 2: To a solution of above intermediate 7 (8 mg, 0.007 mmol) and N-succinimidyl 6-maleimidohexanoate (3.1 mg, 0.010 mmol) in THE (0.20 mL) and DMF (0.10 mL) was added N,Ndiisopropylethylamine(2.5 uL, 0.014 mmol) dropwise. The reaction mixture was allowed to stir at rt for 1 h. The reaction mixture was then concentrated to dryness and the crude residue was purified by reverse phase flash column chromatography (0-100% ACN/aqueous ammonium acetate (10 mM)) to provide title compound 8 as the ammonium salt (4.2 mg, 0.003 mmol, 42% from 6). Nuclear magnetic resonance (NMR) spectra were recorded in the solvent reported on a 400 MHz Bruker spectrometer using residual solvent peaks as the reference.

CDN-linker characterization with ¹H NMR shows (400 MHz, CD30D) δ 8.70 (s, ¹H), 8.40 (br, d, J=8.0 Hz, 2H), 7.69 (d, J=8.0 Hz, 1H), 7.52-7.50 (m, 3H), 7.41-7.39 (m, 1H), 7.23 (br, 3H), 6.84 (d, J=4.0 Hz, 1H), 6.77 (s, 2H), 6.16 (br, s, 1H), 5.59 (br, s, 1H), 5.49 (s, 1H), 5.04 (br, 3H), 4.78 (br, 3H), 4.50 (m, 1H), 4.31-4.24 (m, 3H), 4.17 (d, J=8.0 Hz, 1H), 4.05 (br, d, J=8.0 Hz, 1H), 3.79-3.77 (m, 1H), 3.45 (t, J=8.0 Hz, 2H), 2.89 (s, 3H), 2.57-2.36 (m, 4H), 2.28 (t, J=8.0 Hz, 2H), 2.12-2.07 (m, 1H), 1.86-1.54 (m, 5H), 1.44 (d, J=4.0 Hz, 3H), 1.33-1.25 (m, 2H), 0.97 (t, J=8.0 Hz, 6H). CDN-linker characterization with ³¹P NMR shows (162 MHz, CD30D) δ 55.05 (s, 1P), 53.11 (s, 1P). CDN-linker characterization with ESI-HRMS shows m/z [M+H]+caled. For C55H67N12018P2S2 1309.3613, observed 1309.3631 Purity>95%.

### Example 2: Nanoparticle Synthesis and Characterization

Formation of electrostatic and conjugated CDN-NPs: Electrostatic CDN-NPs were generated by mixing equal volumes of CDN at 0.05 mg mL-1 and pBAE-CR3 polymer at 5 mg mL¹ in sodium acetate buffer (AcONa) at 12.5 mM, followed by 10 min incubation at room temperature (RT). Next, this mixture was nanoprecipitated with two volumes of PBS for the formation of discrete nanoparticles. Conjugated TCDN2 NPs (CDN-NP) were prepared by mixing 25 μL of pBAE-CR3 polymer (100 mg/mL), 20 μL pBAE-TCDN-2 (4 mg/mL) and 5 μL of pBAE-AF647 (2 mg/mL) in DMSO. Then, 450 μL of AcONa at 12.5 mM was added to the polymer solution and mixed by pipetting, followed by 10 min incubation at RT. For the formation of CDN-NPs, this mixture was nanoprecipitated with 2 mL of PBS. Finally, 230 μL of NHS-PEG (2 kDa, Laysan Bio Inc.) (10 mg/mL) were added to the nanoparticles and reacted overnight at room temperature. The final NPs were purified and concentrated using centrifugal filtration (10 kDa MWCO) and filtered through a sterile 0.22 μm membrane.

Biophysical characterization of CDN-NP: The size and surface charge were determined by dynamic light scattering (DLS) and zeta potential, respectively. Approximately 100 μL of CDN-NPs were diluted with 900 μL of PBS and analyzed using a Zetasizer Nano ZS equipped with a He—Ne laser (λ=633 nm) at a scattering angle of 137 (Malvern Instruments Ltd, United Kingdom). Electrostatic CDN formulations were further characterized by agarose gel retardation assay. To assess CDN complexation, different fluorescent CDN (Ap-8-Fluo-AETGp) to polymer ratios (w:w) between 10:1 and 400:1 were studied. pBAE-CR3-CDN nanoparticles were freshly prepared and loaded in 4% E-Gel 31 Precast Agarose Gels (Thermo Fisher), run following the manufacturer's instructions, and visualized in fluorescence mode.

Quantification of CDN: CDN content was determined by liquid chromatography with tandem mass spectrometry (LC-MS-MS). CDN-NPs were incubated with an equal volume of papain digestion solution at 37° C. for 24 h. Next, 40 μL of previous solution was mixed with 300 μL of 0.1% (v/v) formic acid in methanol containing 150 nM carbutamide (internal standard) for 10 min prior to LC-MS-MS analysis.

CDN-NPs stability studies: CDN-NPs were diluted in PBS and changes in their size and polydispersity were determined using DLS over seven days. To determine the stability of CDN-NPs in plasma, CDN-NPs were incubated in mouse plasma at 37° C. and free CDN was quantified by LC-MSMS at different time points as previously described.

### Example 3: Mechanistic and Activity Studies

Cell Lines: Mus musculus skin melanoma (B16-F10, and B16-F10 STING KO from ATCC), Mus musculus colon fibroblast (CT26, from ATCC), Mus musculus mammary gland (4T1, from ATCC) were maintained in Dulbecco's minimum essential medium (DMEM) supplemented with 10% (v/v) fetal bovine serum (FBS), 100 U/mL penicillin and 100 μg/mL streptomycin, 2 mM L-glutamine. RAWLucia™ ISG Cells and RAW-Lucia™ ISG KO-STING cells (InvivoGen) were similarly maintained with the addition of 100 μg/mL Normocin™ and Zeocin™. Human monocyte THP-1 Dual™ cells and THP-1 Dual™ KO-STING cells (InvivoGen) were maintained in RPMI 1640 supplemented with 10% (v/v) FBS, 2 mM L-glutamine, 25 mM HEPES, 100 μg/mL Normocin™, Zeocin™, 10 μg/mL Blasticidin™ (InvivoGen) and 100 U/mL penicillin and 100 μg/mL streptomycin. All cell lines were maintained in a humidified incubator at 37° C., 5% CO2.

Mouse care and experimentation: Female wild type C57BL/6 and BALB/c mice (6-8 weeks old) were purchased from Charles River. C57BL/6J-Stinglgt/J (goldenticket) mice were purchased from The Jackson Laboratory). Animal research and veterinary care was performed at the Hale Building for Transformative Medicine, the Koch Institute for Integrative Cancer Research at Massachusetts Institute of Technology (MIT), and at Takeda Boston under the protocol approved for this study by the Institutional Animal Care and Use Committee (IACUC).

BMDC and BMDM isolation: The tibias and femurs of female wild type or goldenticket C57BL/6 mice (aged 8-12 weeks) were isolated and flushed to harvest bone marrow and to obtain a progenitor cell population. To generate BMDMs, 4-6×10⁶ bone marrow cells were cultured in non-tissue culture treated T175 flasks with 25 mL of DMEM/F12 supplemented with 10% (v/v) FBS, 1% (v/v) P/S, 5% (v/v) GlutaMAX and recombinant murine macrophage-colony-stimulating factor (M-CSF, 20 ng/mL). The flasks were supplemented with additional medium on day 3 and day 6. To generate BMDCs, 2×10⁶ bone marrow cells were added to non-tissue culture-treated petri dishes and cultured in 10 mL of RPMI-1640 supplemented with 10% (v/v) FBS, 1% (v/v) and recombinant murine granulocyte-macrophage colony-stimulating factor (GM-CSF, 20 ng/mL), 10 mL more of which was added on day 3. On day 6 and every other day thereafter until use, 10 mL of consumed medium was centrifuged and replaced with 10 mL of fresh medium. To collect the cells for use in experiments, BMDMs were detached using Accumax and BMDCs were loosely adherent and could be collected by simple washing.

In vitro evaluation of CDN-NP activity: Human monocyte THP-1 Dual™ cells, THP-1 Dual™ KO-STING cells, RAW-Lucia™ ISG cells and RAW-Lucia™ ISG KO-STING cells were seeded in 96-well plates at 1×10⁵ cells per well and incubated with the CDN-NP or free CDN at CDN concentrations ranging from 0-500 nM. At 24 h post treatment, interferon regulatory factor (IRF)3 activity was examined using the QUANTI-Luc™ reagent (InvivoGen) and NF-κB activity (only THP-1 Dual™ cells) was determined using the QUANTI-Blue™ reagent (InvivoGen) according to the manufacturer's instructions.

Cell Viability: Cell viability was assessed using the MTS assay (Promega) as instructed by the manufacturer. After 24 h treatment with varying CDN nanoparticle formulations, MTS reagent was added to the cells to achieve a final MTS concentration of 20% (v/v). Cells were incubated at 37° C., 5% CO2 for up to 2 h and absorbance was measured at 490 nm using a multimodal plate reader (TECAN).

Cell internalization studies: To quantify the relative avidity for CDN-NPs, THP-1 Dual™ cells, RAW-Lucia™ ISG cells, B16-F10, CT26, 4T1, BMDCs and BMDMs were seeded at 1×10⁵ cells per well and incubated with fluorescently labeled CDN-NP at CDN concentrations ranging from 0-1000 nM. After 2 h, excess NPs were removed, cells were washed, trypsinized and fixed with 4% (v/v) paraformaldehyde for 10 min. Fluorescent NPs were detected using flow cytometry using a 640 nm excitation laser and 670/30 nm filter configuration on a BD LSRFortessa cell analyzer.

Assessment of primary immune cell activation: BMDCs or BMDMs were seeded in 24-well plates at 2×10⁵ cells per well and incubated with the CDN-NP or free CDN at equal CDN concentrations ranging from 1-100 nM. NPs that did not contain CDN (Empty-NP) were also used at the equivalent polymer concentration. After 24 hours, the supernatant was removed and cells were then collected, washed, stained, fixed, and analyzed by flow cytometry. The following antibodies were used: CD45 BV785 (clone 30-F11), CD11b BV421 (clone M1/70), F4/80 BUV395 (clone T45-2342), MHCII BV605 (clone M5/114.15.2), CD11c BV421 (clone N418), CD86 BB515 (clone GL-1), CD80 APC (clone 16-10A1), and CD206 PE (clone C068C2). Live cells were gated using LIVE/DEAD™ (Thermo Fisher) near-IR (cat. no. L34976). In addition, IFN-0, IL-6, and TNF-α were analyzed from the supernatant using a custom Legendplex™ panel (BioLegend) as per the manufacturer's instructions.

Cellular transfer of CDN-NPs: Wild type and STING KO B16-F10 cells in a T-75 tissue culture flask at a confluency of 80-90% were treated with free CDN, empty NP, or CDN-NPs at equivalent doses for 4 hours. After this time, the cells were washed, trypsinized and seeded in 24-well plates at a density of 1×10⁵ cells/well. Once the cells were attached, 1×10⁵ immune cells (wild type and STING KO THP-1 Dual™, RAW-Lucia™ ISG, BMDC and BMDM) were added to the wells and incubated for 24 hours. In the case of the immortalized reporter cell lines, the supernatant was analyzed according to the manufacturer's instructions. For experiments involving primary immune cells, the cells were then collected, washed, stained, fixed and analyzed by flow cytometry. For in vivo studies, B16-F10 cells or B16-F10 STING KO cells were treated identically as above prior to subcutaneous implantation in the right flank of female C57BL/6 mice (5×10⁵ cells/injection in 100 μL HBSS). Cells treated with Empty-NP alone at the equivalent polymer concentration were used as a control. Tumors were monitored until the humane endpoint was reached.

Pharmacokinetics of CDN-NPs: Non-tumor bearing 6-8-week-old female C57BL/6 mice were injected i.v. (100 μL per mouse) with fluorescent CDN-NPs and whole blood was collected 0.25, 0.5, 1, 3, 6, and 24 h post injection in Eppendorf tubes containing heparin though submandibular bleed (for early time points) or cardiac puncture (for late time points). Plasma was obtained by centrifugation for 20 mins at 10000×g. Fluorescent CDN-NPs were quantified using a 640 nm excitation and 680 emission wavelength by plate reader and CDN was also quantified by LC-MS/MS.

Biodistribution and cellular localization of CDN-2 NPs: C57BL/6 mice bearing B16-F10 tumors (100-200 mm³) were injected i.v. (100 μL per mouse) with fluorescent CDN-NPs and euthanized at 6 h, 24 h, and 48 h post injection. Tumor, tdLN, liver, lung, heart, kidney, and spleen were collected, imaged using an In Vivo Imaging System (IVIS), and digested using Precellys lysing kits (Bertin Instruments) following the manufacture instructions. Fluorescent CDN-NPs distribution was quantified by reference to a standard curve and background fluorescence was removed by subtracting baseline fluorescence values of tissue lysates from PBS-treated mice. CDN was also quantified by LC-MS-MS as described above. To determine the cellular populations that contained CDN-NPs, mice bearing B16-F10 tumors were treated as described above and were euthanized 4 h and 24 h post injection. The tumor, tdLN, spleen, and liver were collected, dissociated into single cell suspensions as described below, stained, fixed, washed, and resuspended in cell staining buffer (BioLegend). The following antibodies were used: CD45 APC-Cy7 (clone 30-F11), CD45 BV785 (clone 30-F11), NK-1.1 BV710 (clone PK136), CD3 BB700 (clone 17A2), CD8a BV421 (clone 53-6.7), CD4 BUV395 (clone GK1.5), CD11b BV421 (clone M1/70), F4/80 BUV395 (clone T45-2342), Ly-6C APC-Cy7 (clone HK1.4), Ly-6G BV661 (clone 1A8), MHCII BV605 (clone M5/114.15.2), CD11c BV421 (clone N418). Live cells were gated using LIVE/DEAD™ (Thermo Fisher) aqua (cat. no. L34966), green (cat. no. L34970) or near-IR (cat. no. L34976).

Stained cells were analyzed by flow cytometry using a BD LSRFortessa™ flow cytometer (BD Biosciences) and all data were analyzed using FlowJo software (FlowJo LLC).

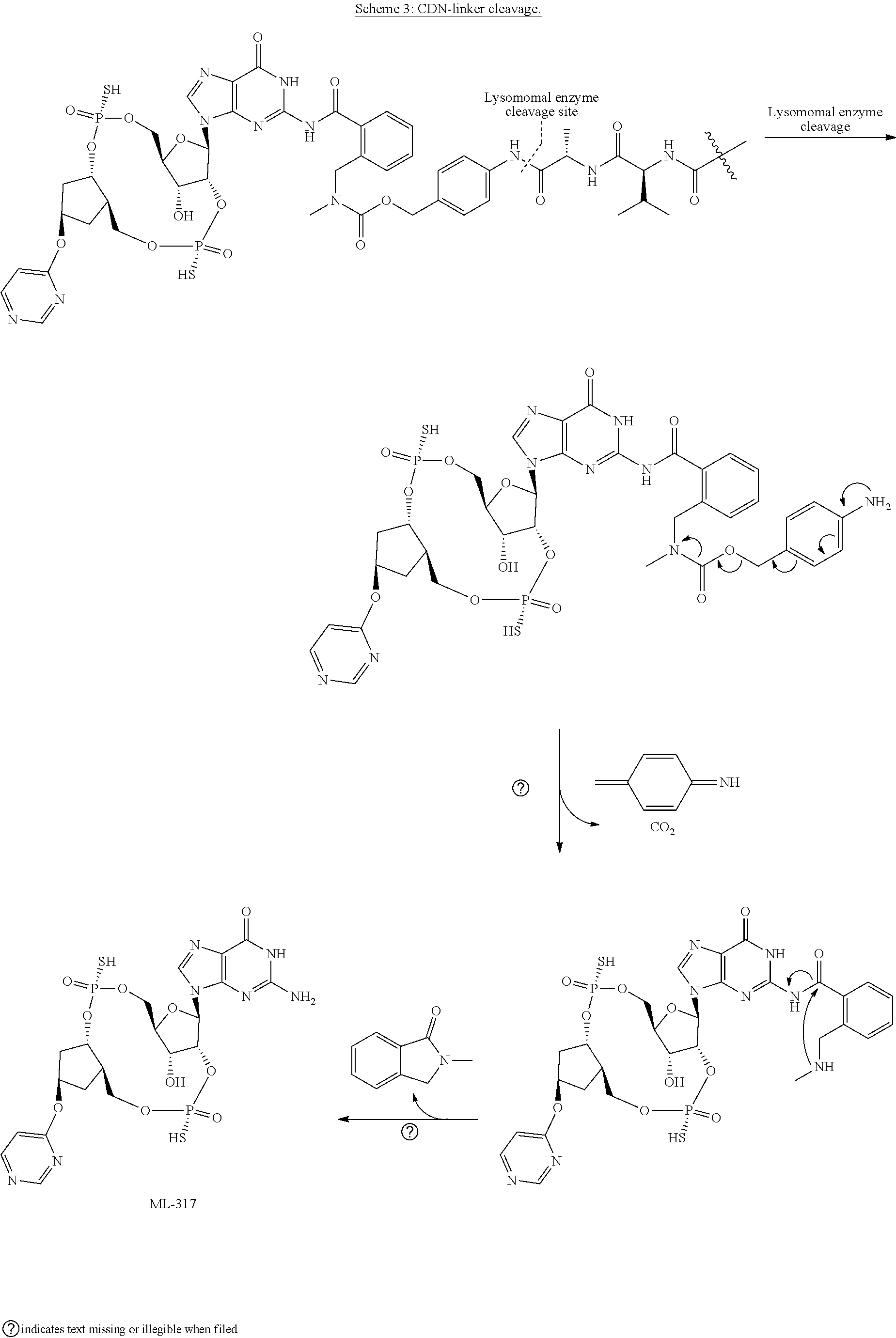

The CDN releasing pathway using Val-Ala dipeptide involves 2-step self-immolating process.

Conjugated CDN-NPs enhance immune-cell activation compared to CDN polyplexes and to free CDN

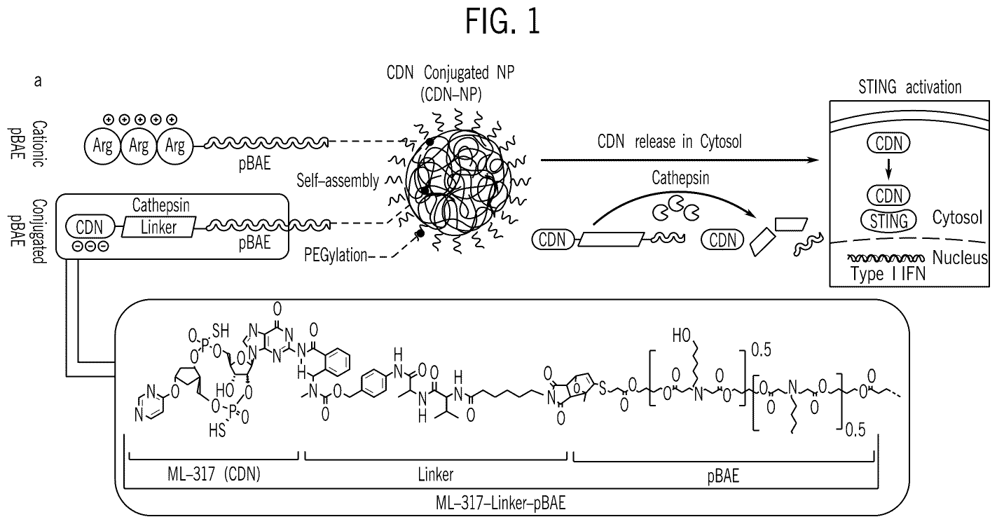

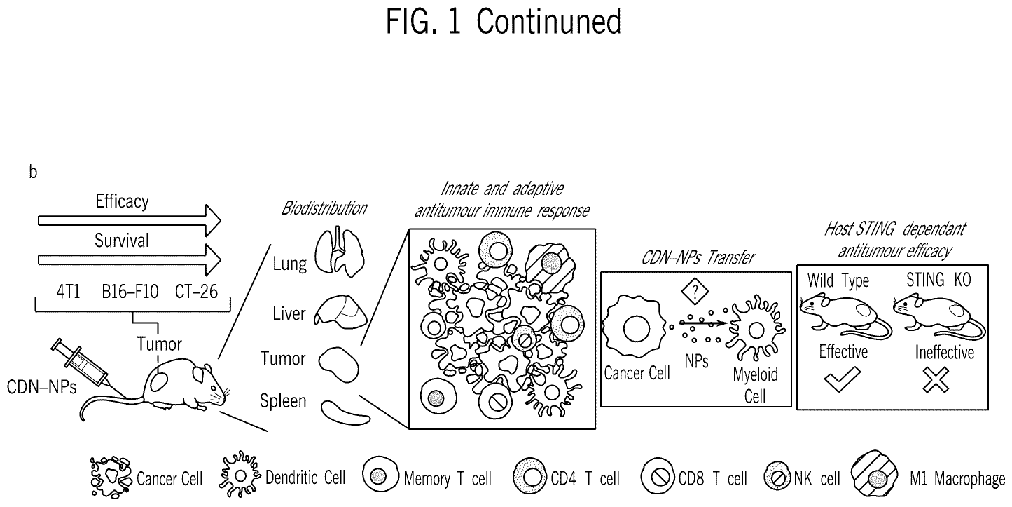

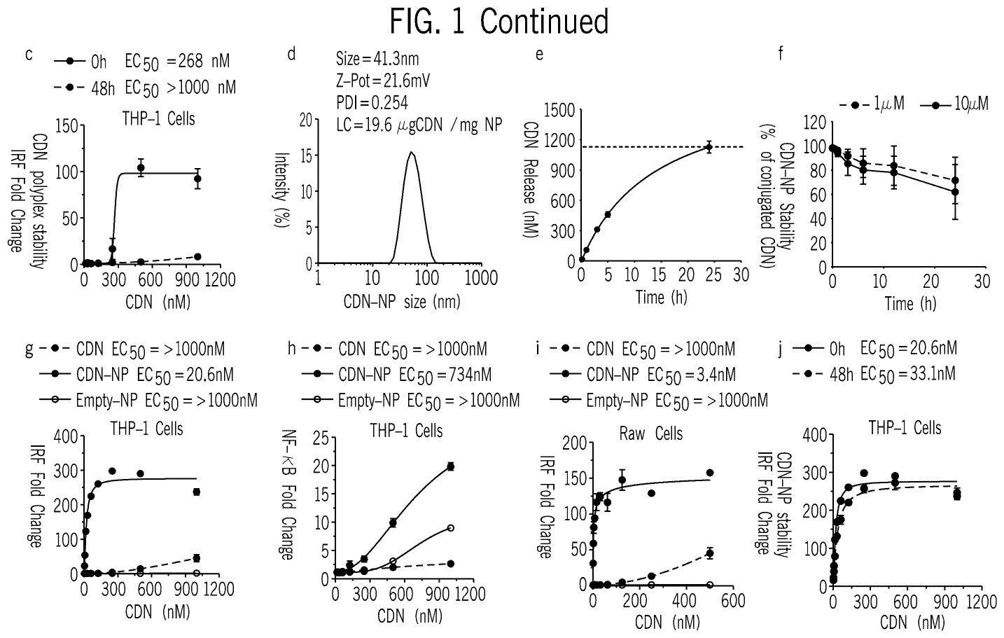

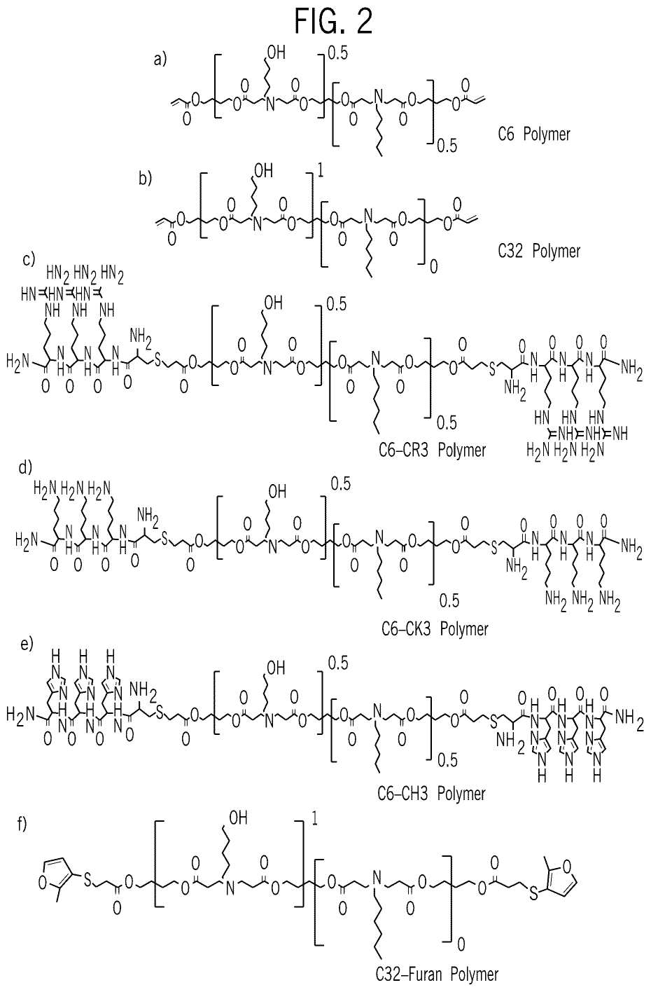

CDNs are small nucleic acid-based molecules which are negatively charged and hence are traditionally electrostatically complexed with cationic polymers. Here, we developed a cationic polymeric NP system based on pBAEs to facilitate covalent conjugation and effective cytosolic delivery of CDN (FIG. 1(a)). We tested our new CDN formulation in various tumor models, and we studied the role of cancer cells and host immunity in the context of CDN therapeutic efficacy (FIG. 1(b)).

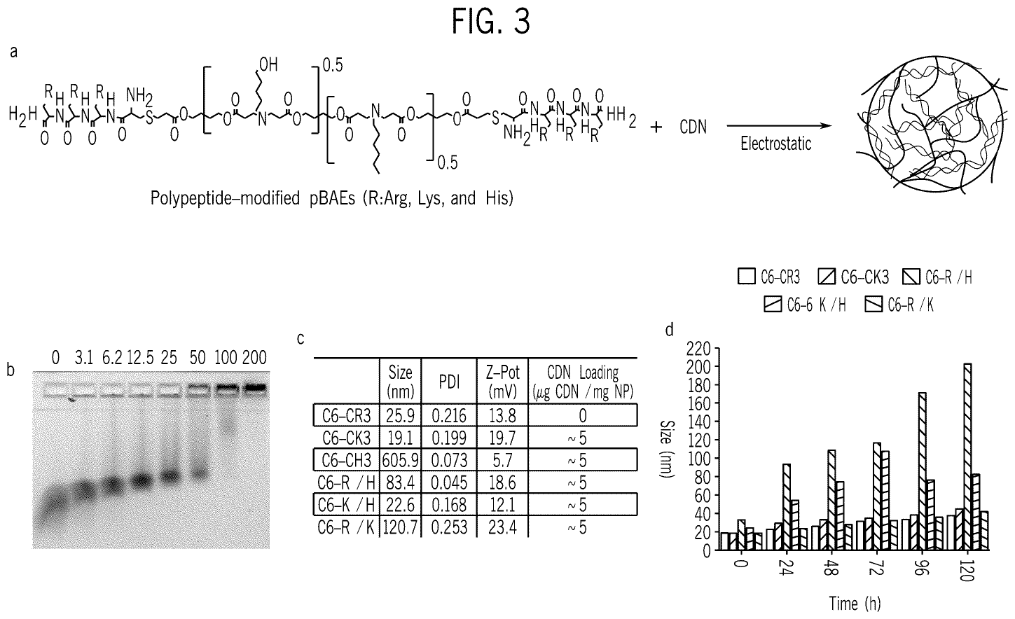

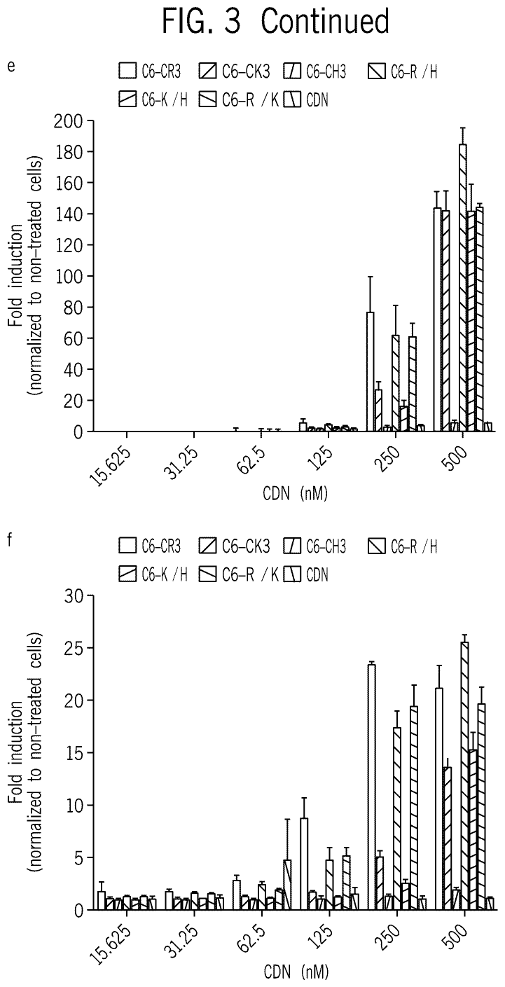

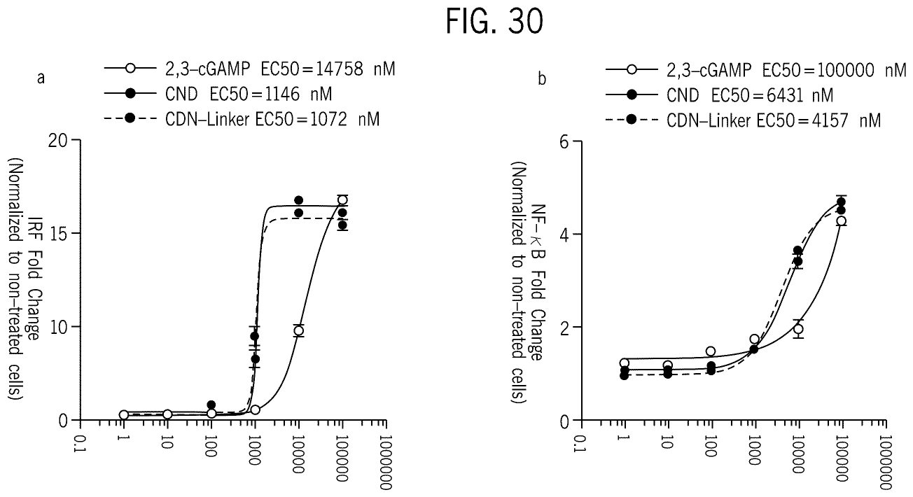

We have previously used polypeptide end-modified polymers to complex anionically charged gene therapies through electrostatic interactions for local and systemic delivery of nucleic acids. Here we leveraged the ability of polypeptide-conjugated pBAE polymers to deliver CDN and stimulate IRF3 and NF-κB responses, both downstream of STING, in reporter human monocytic THP-1 Dual™ cell line. We found that pBAEs end-modified with arginine polypeptides (C6-CR3) complexed with CDN showed the highest levels of IRF3 and NF-κB activation in THP-1 Dual™ lines, achieving a maximum effect (Emax) of more than 100-fold and 20-fold increase when compared to free CDN, respectively (FIGS. 3(e), 3(f)). C6-CR3 NPs were stable in PBS (up to 120 hours) (FIG. 3(d)), however, they failed to activate IRF3 after 48 hours of incubation in PBS due to the low molecular weight of the CDN and weak negative charge density (only two phosphate groups per molecule), limiting its utility for systemic administration (FIG. 1(c)). To overcome this, we modified the STING agonist ML-317 (CDN) containing a cathepsin-cleavable linker and terminal maleimide group (FIG. 1(a) and Scheme 2), allowing its covalent conjugation to pBAE polymer. The CDN modification did not affect its in vitro activity compared to unmodified CDN (FIG. 30). The cathepsin-cleavable linker was incorporated to facilitate lysosomal proteolysis of the CDN into its free form within the cell via a two-step self-immolating process (Scheme 3).

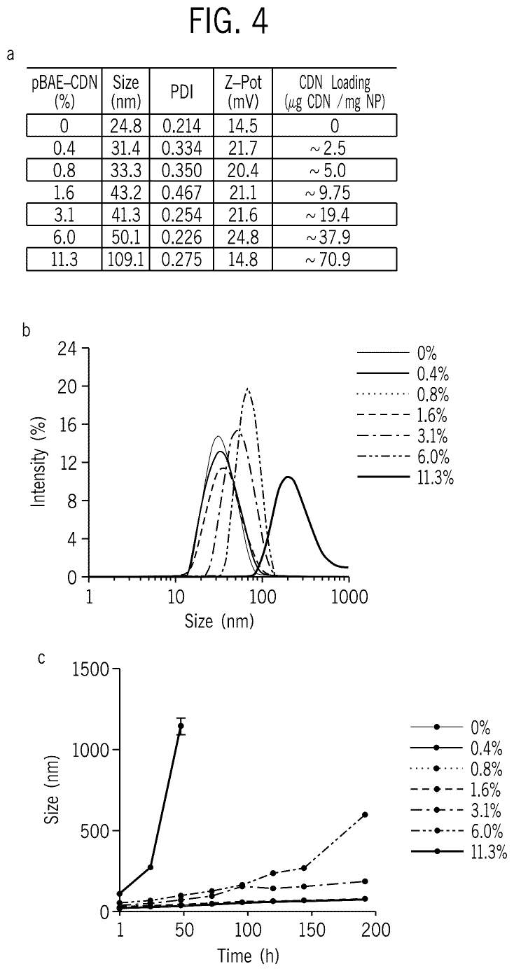

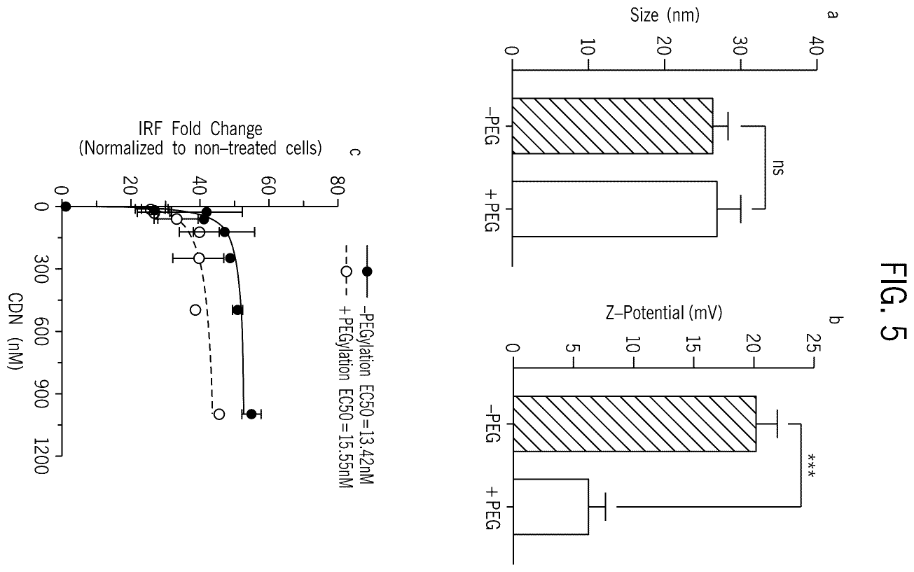

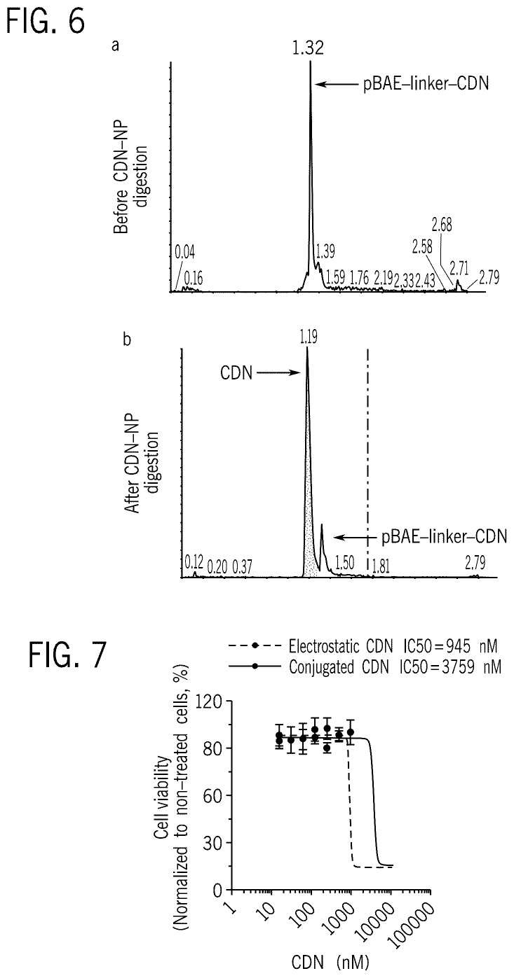

To conjugate the CDN, pBAE acrylate end groups were functionalized with 2-methyl-3-furanthiol (Scheme 1), allowing a Diels-Alder reaction between the CDN maleimide group and pBAE polymer furan group. To achieve high encapsulation efficiencies, two maleimide-modified CDN molecules were conjugated per pBAE polymer chain. The resultant NPs were self-assembled through the electrostatic interaction between the arginine-modified pBAE (C6-CR3) and the CDN-modified pBAE polymer (ML-317-Linker-pBAE) to form CDN-NPs (FIG. 1(a)). NPs containing up to 3.1% (w/w) of ML-317-Linker-pBAE polymer can be formed without compromising their stability, as assessed by DLS. Further increase in ML-317-Linker-pBAE content per mg of total polymer resulted in an increase in nanoparticle size and a decrease in its stability (FIG. 4). CDN-NPs had an average size of 20-50 nm with a positive surface charge of 20 mV (FIG. 1(d)) and were PEGylated to enhance their circulatory half-life. The PEGylation of CDN-NPs was confirmed by the decrease of overall surface charge (from 20.2 mV to 6.3 mV) (FIG. 5(b)). In addition, PEGylation did not significatively affect CDN-NPs in vitro activity (FIG. 5(c)). To affirm the successful conjugation of CDN and the responsiveness to enzymatic cleavage, we quantified the CDN released from CDN-NPs upon exposure to papain, by LC-MS/MS. CDN-NPs were found to contain 18.1±0.9 μg CDN/mg polymer, giving a reaction yield of 45.3±2.4% and all the CDN was released upon enzymatic incubation for 24h (FIGS. 1(e), 6), demonstrating successful conjugation and subsequent cleavage of the CDN. Conjugated CDN-NPs enabled quadruple the CDN content compared to electrostatic CDN-NPs (˜5 μg CDN/mg NP), resulting in greater cell tolerance (IC50=3759 nM for conjugated CDN formulations and IC50=945 nM for electrostatic CDN formulations) (FIG. 7). In addition, we confirmed that CDNNPs are stable in mouse plasma and found that less than 20% of the total CDN was released in 12 h (FIG. 1(f)).

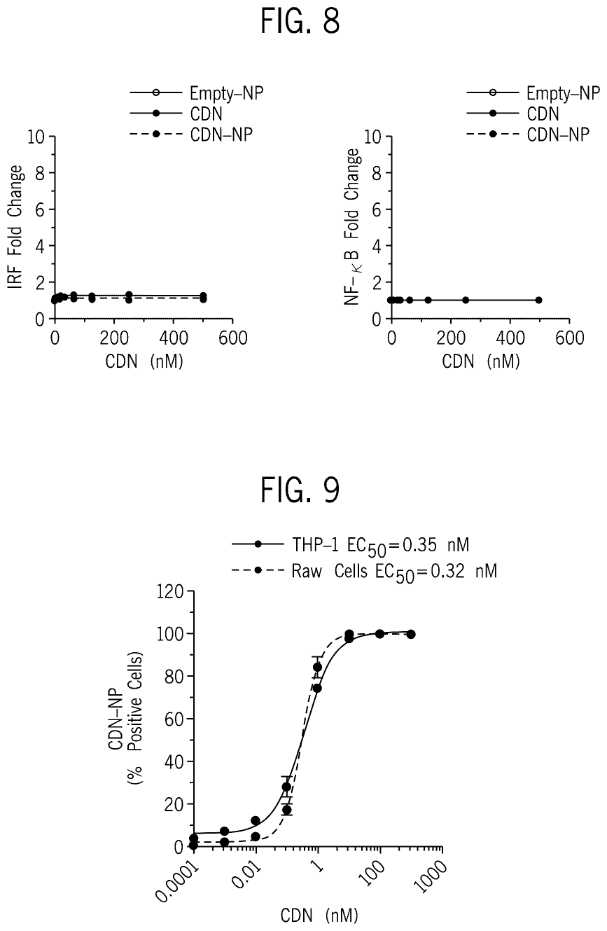

Next, we assessed the ability of CDN-NP to deliver CDN and stimulate IRF3 and NF-κB responses in THP-1 reporter cell line (FIGS. 1(g), 1(h), 30). CDN-NPs resulted in markedly higher IRF3 activation than free CDN in THP-1 Dual™ cells [half-maximum effective concentration (EC50)=20.6 nM and 1146 nM for CDN-NPs and free CDN respectively]. Interestingly, CDN conjugated formulations achieved the highest IRF3 responses in THP-1 Dual™ cells, up to a 13-fold increase when compared to electrostatic CDN formulations (EC50=20.6 nM for conjugated CDN-NPs and EC50=268 nM for electrostatic CDN-NPs). Similar trend was observed for NF-κB response, where CDN-NP resulted in higher NF-κB activation than free CDN or Empty-NPs (FIG. 1(h)). We did not observe activation of IRF3 or NF-κB in STING-KO THP-1 Dual™ cells (FIG. 8) demonstrating that the activation was STING dependent. A comparable enhancement in CDN-NP potency was observed in RAW-Lucia™ ISG cells (EC50=3.4 nM) compared to free CDN (EC50>1000 nM) (FIG. 1(i)). In addition, CDN-NPs remain active following 48 hours of incubation in PBS, demonstrating the stability of CDN-NPs (FIG. 1(j)).

To investigate the cellular uptake of CDN-NPs, THP-1 Dual™ and RAW 264.7 cells were incubated with fluorescently labelled CDN-NP for 4 h in complete medium (FIG. 9). CDN-NPs showed more than 90% uptake at 1 nM CDN. EC50 of 0.35 nM and 0.32 nM was observed in THP-1 Dual™ and RAW 264.7 cells, respectively, confirming that CDN-NPs were avidly taken up by human and murine monocytes/macrophages in vitro. This led to potent STING activation at low nanomolar (nM) concentrations as free CDN enters cells far less effectively than CDN-NPs.

CDN NPs uptake by immune cells, either directly or following transfer from cancer cells, enhance the immunostimulatory activity of CDN

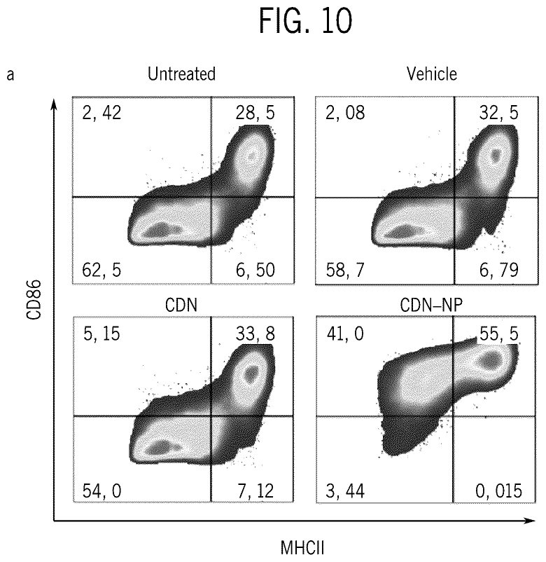

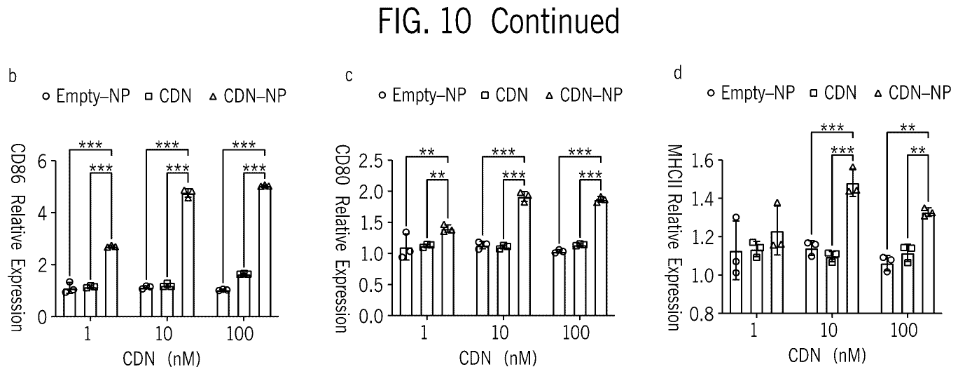

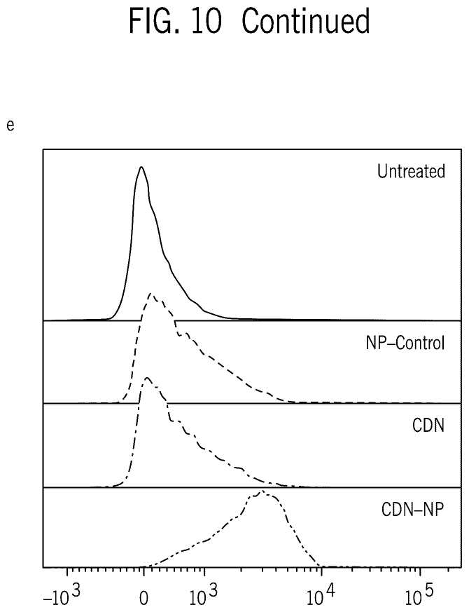

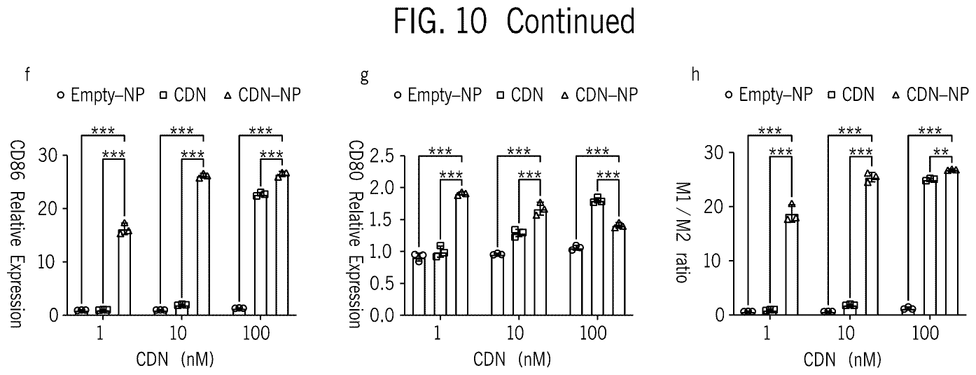

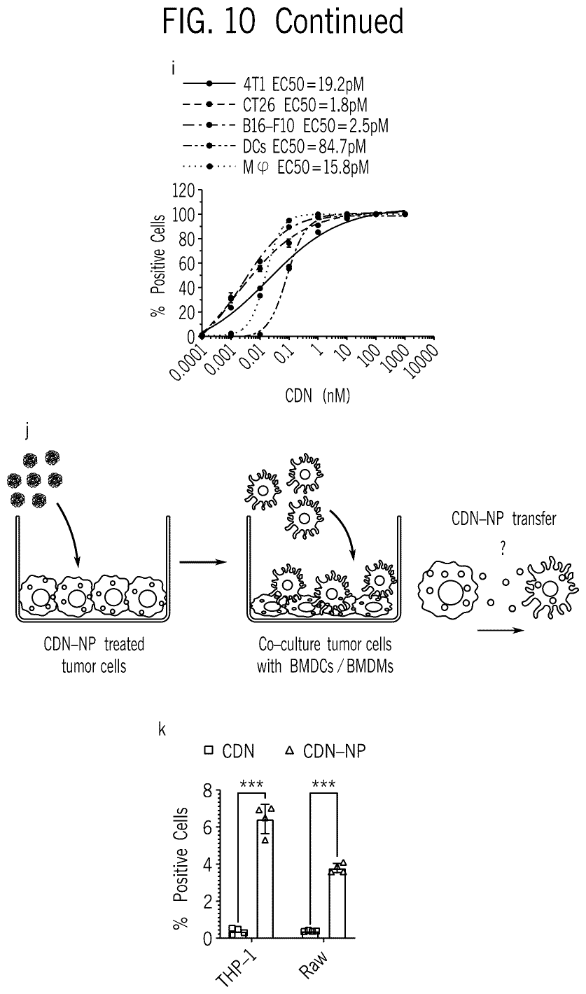

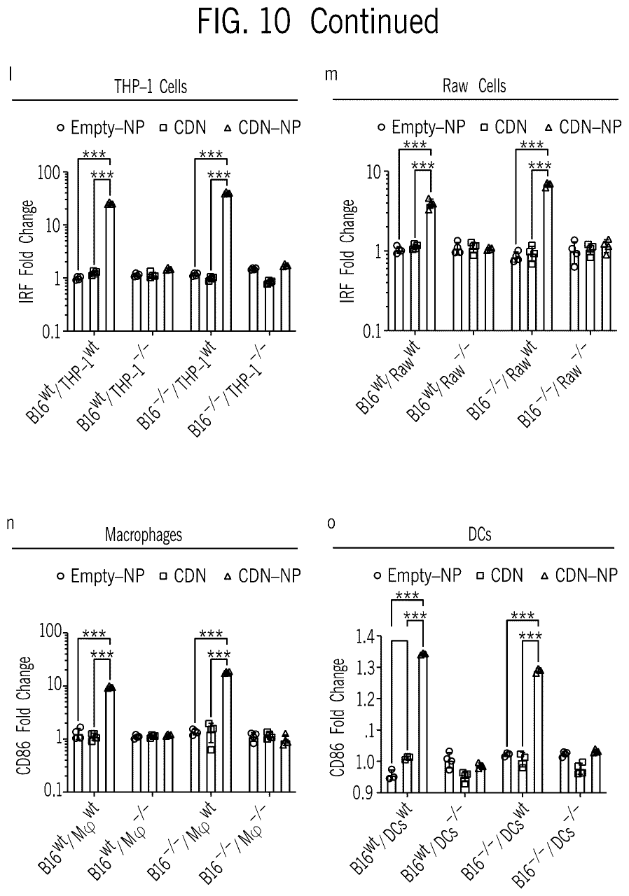

We evaluated the capability of CDN-NPs to activate dendritic cells (DCs) and macrophages. Bone marrow derived dendritic cells (BMDCs) were treated for 24 h and the expression of activation markers CD80, CD86, and major histocompatibility complex class II (MHC-II) was analyzed by flow cytometry and reported as Mean Fluorescence Intensity (MFI). A dose dependent increase in CD86 expression was observed in CDN-NP treated groups, achieving more than 5-fold increase when compared to free CDN at a 10 nM dose (FIGS. 10(a), 10(b)). Marked increases in both CD80 and MHCII were also observed in the CDN-NP treated groups (FIGS. 10(c), 10(d)). These results suggest that CDN-NPs stimulate DCs maturation and activation. Similar results were obtained using bone-marrow derived macrophages (BMDMs). Low CDN-NP doses (1 nM) enhanced CD86 expression by 15-fold compared to non-treated macrophages, while a high dose of free CDN (100 nM) was required to achieve the same degree of CD86 upregulation (FIGS. 10(e), 10(f)). No increase in CD86 expression was observed for an Empty-NP. A comparable profile was observed when the pro-inflammatory M1-like and anti-inflammatory M2-like ratio was studied (FIG. 10(h)). A 2-fold increase in CD80 expression was observed at low CDNNP doses compared to free CDN (FIG. 10(g)).

We previously confirmed the capability of CDN-NPs to be internalized by immune cells (FIG. 1(j)). However, due to the cellular heterogenicity in the TME, we compared the ability of CDN-NPs to efficiently deliver CDN to immune cells and murine melanoma, breast, and colon cancer cells. CDNNPs showed the highest level of cellular uptake in B16-F10 and CT-26 cells, achieving up to a 20-fold and 10-fold increase compared to DCs and BMDMs, respectively (FIG. 10(i)). These results suggest that cancer cells may compete with the target immune cells for NP internalization. Interestingly, cancer cells are known to produce CDN molecules that are transferred via gap junctions to tumor associated DCs and macrophages, inducing type I IFN production. We thus hypothesized that cancer cells can act as a reservoir of CDN-NPs, transferring CDN-NPs to DCs or macrophages over time. To investigate this phenomenon, we determined the presence of transferred CDN-NPs in to THP-1 and Raw cell lines after coculturing them for 24 h with tumor cells pre-treated with free CDN or CDN-NPs at equivalent doses (FIG. 10(j)). Results showed that 6.4±0.4% of THP-1 cells and 3.8±0.1% of Raw cells contained CDN-NPs transferred from cancer cells (FIG. 10(k)). We also evaluated the IRF3 activation of STING wild type (WT) and knock out (KO) monocytic cell lines (THP-1 Dual and Raw cells) and CD86 activation marker in primary immune cells from the transferred CDN-NPs. We found that IRF3 is activated in STING WT THP-1 Dual™ (THP-1^{wt}) and Raw (Raw^{wt}) cells, but not in STING KO THP-1 Dual™ (TIP-1^{-/-}) or STING KO Raw (Raw^{-/-}) cells co-cultured with tumor cells (either STING WT (B16^{wt}) or STING KO B16-F10 (B16^{-/-}) cells) pre-treated with CDN-NPs (FIGS. 10(l), 10(m)). A similar trend was obtained using STING WT BMDCs (DCs^{wt}) and BMDMs (MF^{wt}), where an increase of activated CD86⁺ cells was observed when cancer cells where pre-treated with CDN-NPs. Also, no CD86 increase was observed when STING KO DCs (DCs^{-/-}) or macrophages (MF^{-/-}) were used (FIGS. 10(n), 10(o)). These results suggest that cancer cells transferred CDN-NPs to immune cells and activate STING pathway in immune cells, acting as a reservoir of CDN-NPs.

### Example 4: In Vivo Studies

In vivo therapeutic efficacy: B16-F10, 4T1, and CT-26 cells were injected subcutaneously into the right flank of 6-8-week-old female mice (of the appropriate background for the cell type) in 100 μL HBSS at densities of 5×10⁵, 1×10⁶, and 1×10⁶ cells/injection, respectively. Approximately 7 days post tumor inoculation (˜50 mm³), mice were subjected to intravenous injection (100 μL) of free CDN or CDN-NP at a CDN dose of 0.5 μg. Mice were injected 3 times with treatments spaced 4 d apart. Groups receiving checkpoint blockade were injected intraperitoneally 24 h post systemic administration with 100 μg anti-PD-1 (clone RMP1.14, Bio X Cell). The tumor size was measured every other day via caliper measurements and the tumor volume calculated using the equation V=L×W×H×(π/6). Body weight was measured contemporaneously with tumor volume. Mice were euthanized when tumors reached a volume of 1000 mm³ or exhibited poor body condition.

Confirmation of STING-dependent antitumor activity: Wild type or goldenticket female C57BL/6 mice (6-8 weeks old) were inoculated subcutaneously with either wild type B16-F10 tumor cells or B16-F10 STING KO tumor cells. When tumors reached ˜50 mm³, mice were injected intravenously with CDN-NPs (100 μL) at a CDN dose of 0.5 μg and were injected a total of 3 times with treatments spaced 4 d apart. Mice were euthanized when the humane endpoint was reached.

STING-dependent therapeutic efficacy in spleen deficient mice: C57BL/6 mice were subjected to splenectomy one-week prior therapeutic efficacy studies. B16-F10 tumors were implanted as described above. 7 days post tumor inoculation (˜50 mm³), mice were subjected to intravenous injection (100 μL) of CDN-NP (0.5 μg) with or without antiPD-1 (100 g) following the same dose regimen described above. Tumor size and survival was monitored until tumors reached a volume of 1000 mm³ or exhibited poor body condition.

Immunophenotyping analysis: Subcutaneous B16-F10 tumors were established in female C57BL6/J mice (6-8 weeks old), as previously described. The treatment groups were: PBS (untreated control), aPD-1 mAb, free CDN, free CDN with aPD-1 mAb, CDN-NPs, and CDN NPs with aPD-1 mAb. CDN formulations (free drug or NP, 0.5 μg CDN) were administered intravenously and aPD-1 mAb (100 μg) was administered intraperitoneally 24 h after CDN treatment. Dendritic cells and macrophages were assessed 48 h after treatment and T cells were analyzed 7 days post-treatment. Tumors were harvested, chopped, and digested in a solution of HBSS supplemented with collagenase I, II, and IV (100 ng/mL) and DNase I (1 g/mL) for 2 h at 37° C. TdLNs and spleens were harvested and mechanically dissociated. Single cell suspensions of tumors, tdLNs, and spleens were filtered through a 40 m nylon cell strainer. Spleen and tumor cells were further treated with ACK Lysing Buffer (Gibco). Cells were washed, filtered through a 40 μm nylon cell strainer, and counted. For intracellular IFN-γ cytokine analysis, 1×10⁶ cells were seeded in a 24-well plate in DMEM containing 10% (v/v) FBS and supplemented with PMA/ionomycin/Brefeldin A cocktail (BioLegend). After 4 h, the cells were washed and stained in 100 μL cell staining buffer (BioLegend). Intracellular staining was performed using an intracellular staining permeabilization wash buffer (BioLegend) The following anti-mouse antibodies were used for flow cytometry were purchased from BioLegend: CD45 APC-Cy7 (clone 30-F11), NK-1.1 BV710 (clone PK136), IFN-γ BV421 (clone XMG1.2), CD279 (PD-1) FITC (clone 29F.1A12), CD45 BV785 (clone 30-F11), CD11b BV421 (clone M1/70), Ly-6C APC-Cy7 (clone HK1.4), Ly-6G BV661 (clone 1A8), CD8a BV421 (clone 53-6.7), CD86 BV510 (clone GL-1), CD80 BV711 (clone 16-10A1), CD206 PE (clone C068C2), MHCII BV605 (clone M5/114.15.2), CD11c APC (clone N418), Siglec H PE (clone 551), and CD44 BV786 (clone IM7).

The following anti-mouse antibodies were purchased from BD Biosciences: CD3 BB700 (clone 17A2), CD4 BUV395 (clone GK1.5), CD8a BUV737 (clone 53-6.7), F4/80 BUV395 (clone T45-2342), CD103 BUV395 (clone M290), CD80 BUV737 (clone 16-10A1), ki67 BV510 (clone B56), CD69 BV605 (clone H1.2F3), and CD62L PE-CF594 (clone MEL-14). Live cells were gated using LIVE/DEAD™ (Thermo Fisher) aqua (cat. no. L34966), green (cat. no. L34970), or near-IR (cat. no. L34976). Stained cells were analyzed by flow cytometry using a BD LSRFortessa™ flow cytometer (BD Biosciences) and all data were analyzed using FlowJo software (FlowJo LLC).

Cytokine analysis: Female C57BL/6 mice (6-8 weeks) bearing B16-F10 tumors were given one intravenous injection of PBS, free CDN, CDN-NP at a dose of 0.5 μg or Empty-NP (non-CDN containing NPs at the equivalent polymer concentration). After 4 h, mice were euthanized, tumors, tdLNs and spleens were harvested and blood was collected, after which plasma was obtained by centrifugation. Tissues were lysed with 0.5% (v/v) CHAPS containing protease and phosphatase inhibitors. Levels of IFN-g, IFN-b, IFN-α (MSD U-Plex interferon combo, cat. no. K15320K-2), IL-1b, IL-2, IL-4, IL-5, IL-6, IL-9, KC/GRO, IL-10, IL-12p70 (MSD Proinflammatory Panel, cat. no. K15045G-2), TNF-α, IL-15, IL-17A/F, IL-27p28/IL-30, L-33, IP-10, MCP-1, MIP-1α and MIP-2 (MSD Cytokine Panel, cat. no. K15245D-2) were measured according to the manufacturer's instructions.

Statistical Analysis: Statistical analyses were carried out using Graph-Pad Prism 9 (GraphPad Software). All data are reported as mean+SEMs. For in vitro experiments, a minimum of n=3 biological replicates were used per condition in each experiment. Pairwise comparisons were performed using Student t-tests. Multiple comparisons among groups were determined using one-way ANOVA followed by a post-hoc test. For in vivo experiments, a minimum of n=5 biological replicates were used per condition in each experiment. Multiple comparisons among groups were determined using Kruskal-Wallis test with uncorrected Dunn's test. Kaplan-Meier survival curves statistical analysis was determined using two-tailed Mantel-Cox test. Differences between groups were considered significant at p-values below P<0.033 (****P<0.0001, ***P<0.0002, **P<0.002, *P<0.033).

### CDN-NPs are Internalized by Immune Cells in the Tumor and in Secondary Lymphoid Organs In Vivo, Producing Anti-Inflammatory Cytokines

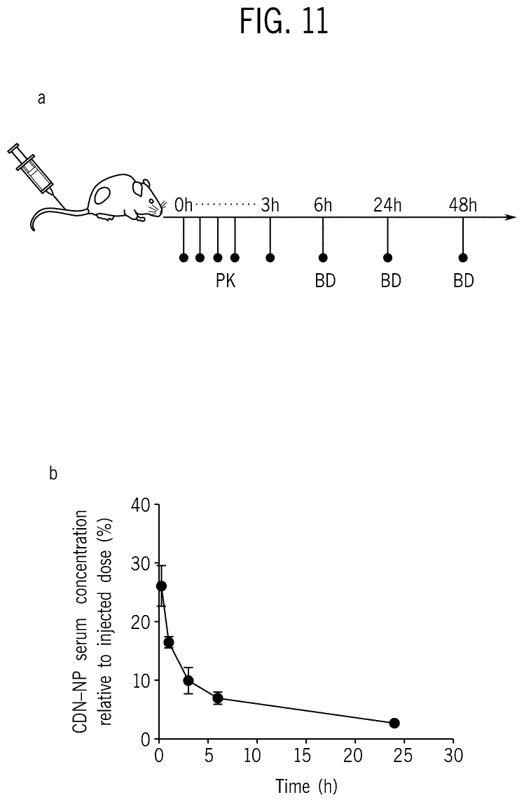

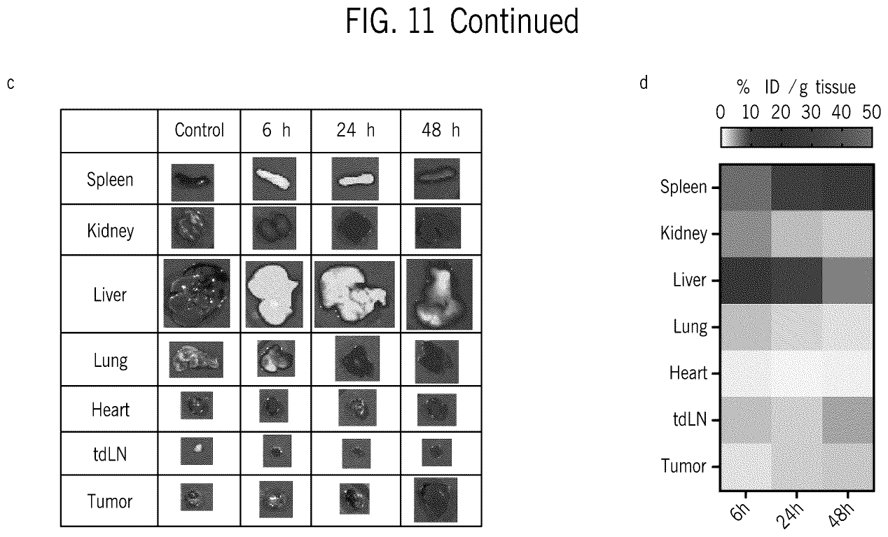

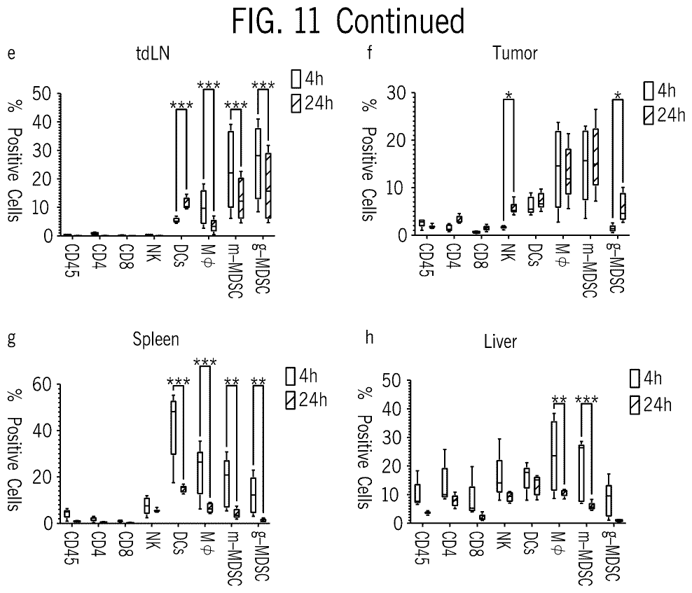

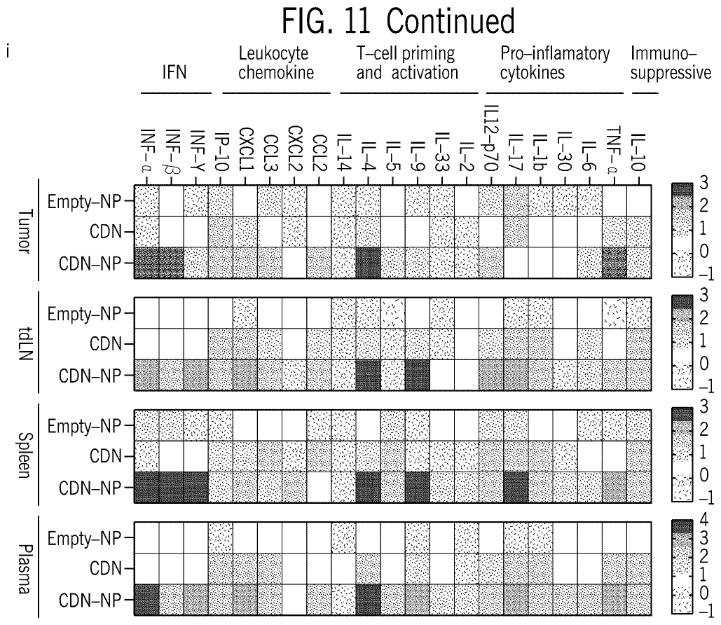

A desirable property of systemically injected formulations, particularly those intended for the TME, is improved pharmacokinetics (PK). To understand the PK of CDN-NPs, mice bearing B16 tumors were injected intravenously with fluorescently labelled CDN-NPs or free CDN. CDN and polymer concentration was quantified by LC-MS-MS and fluorescence, respectively. We observed that free CDNs were below the detection limit of 5 nM in plasma 15 min after intravenous administration. In contrast, CDN derived from CDN-NPs was readily detected up to 1 h post administration, where plasma concentrations of 50 nM (16.2±0.5% of the initial dose) were observed (Table 1). We also measured the fluorescence of the NPs in the plasma, and after 1 h post administration about 16.5±1.7% of the initial injected dose was detected in the plasma, and 2.7±0.6% of NPs were still in circulation 24 h post-administration (FIG. 11(b)). These results confirm the integrity of our CDNNP as similar percentage of polymer and of CDN was observed 1 h post intravenous administration. To understand the biodistribution BD of CDN-NPs, mice were intravenously injected with fluorescent formulations and organs were analyzed at different time points. NPs were found to preferentially accumulate in the liver (13.1±2.4% ID), spleen (4.2±0.2% ID), and lung (0.6±0.2% ID), at an early time point (6 h post-administration) which is consistent with the BD profile observed using arginine modified pBAE NPs42. However, the mean fluorescence intensity (MFI) rapidly decreased in those organs at 48 h post-administration while it slightly increased in the tdLN and the tumor (FIGS. 16, 11(c)). In line with these observations, quantification of injected dose per gram of tissue revealed CDN-NPs were predominantly taken up by the spleen and the liver (FIGS. 11(d), 11(e)). Relative to the other organs, CDN-NPs accumulated the most in the spleen. Indeed, NP properties such as size and surface charge are known to influence the deposition of NPs in vivo. The size of CDN-NPs is highly consistent with bestowing hepatic or splenic tropism, which agrees with our observations.

TABLE 1
Pharmacokinetic analysis of CDN-NPs or free CDN
upon i.v. administration by LC-MS-MS
(n = 3 biologically independent samples).
Total CDN and percentage of Injected Dose
(ID) are represented in this table.

| Time | CDN-NP | | Free CDN | |
|---|---|---|---|---|
| (min) | CDN (nM) | ID (%) | CDN (nM) | ID (%) |
| 15 | 124 ± 42 | 41.2 ± 14 | <5 nM | — |
| 30 | 66 ± 12 | 21.8 ± 4 | — | — |
| 60 | 49 ± 2 | 16.2 ± 0.5 | — | — |
| 180 | <5 nM | — | — | — |

The BD profile of CDN-NPs combined with the critical role of immune cell populations in mounting a STING-mediated antitumor response, prompted us to understand which cell populations were internalizing the CDN-NPs. CDN-NP internalization kinetics and trafficking post intravenous administration were studied at two different time points, 4 and 24 hours, in the spleen, liver, tdLN, and the tumor (FIGS. 11(f)-11(i)). In the spleen, tdLN, and tumor, NPs were principally internalized by DCs, macrophages, granulocytic cells, or monocytic cells. In the spleen, and to a lesser extent in the liver, the percentage of DCs and macrophages containing NPs [demarcated as % NP positive (NP+)] decreased at 24 h compared to 4 h post administration. Interestingly, the converse was observed in the tdLN whereby an increase in DCs was witnessed at 24 h (5.4±1.1% NP+ at 4 h and 11.2±2.3% NP+ at 24 h). A decrease in other myeloid populations was similarly seen 24 h post administration in the tdLN as was seen in the spleen and liver. These data suggest that the immune cell populations in the spleen and liver could be directly involved in the antitumor efficacy of CDN-NPs whether by assisting in the priming of T cells to induce antitumor functionality, by secretion of cytokines or even their migration to the tumor or the tdLN.

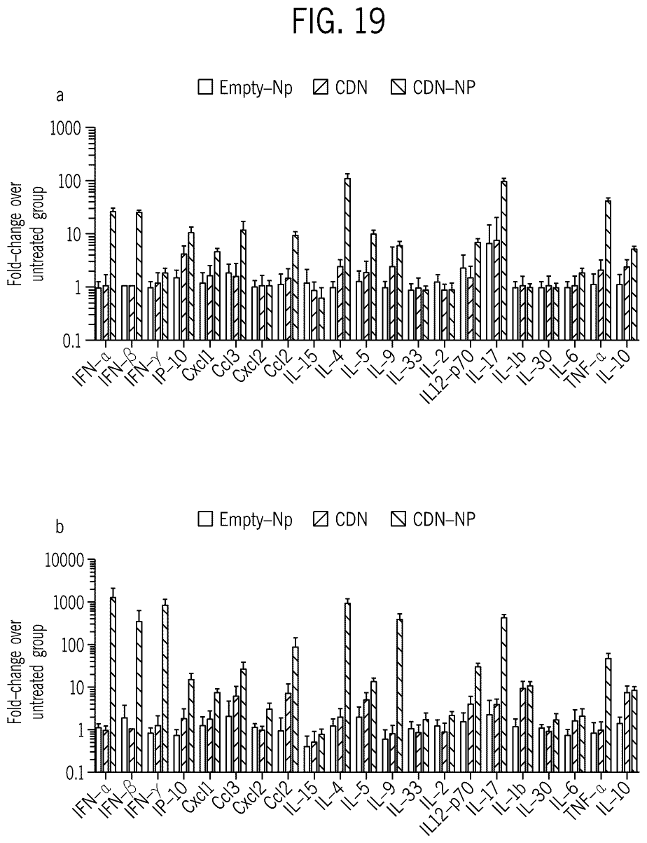

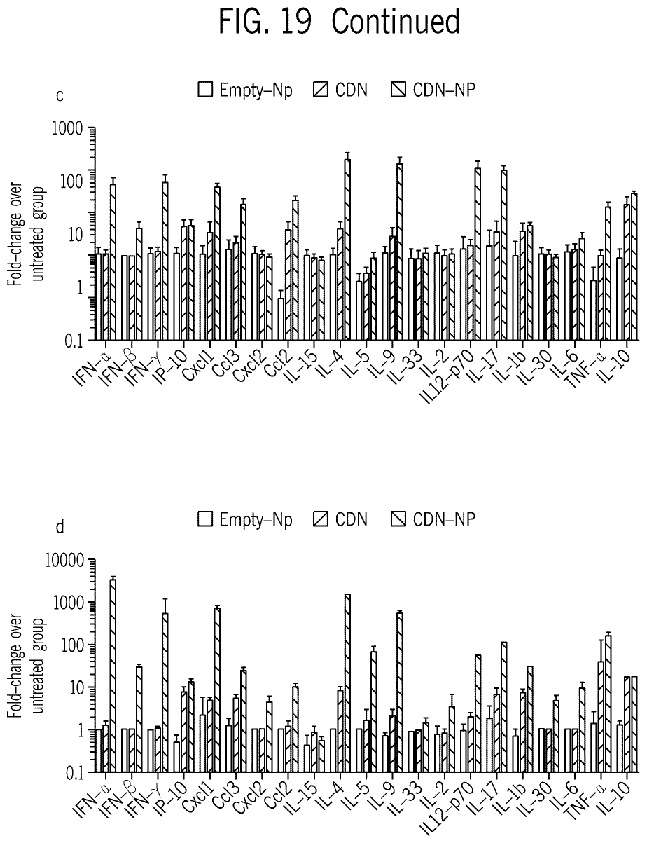

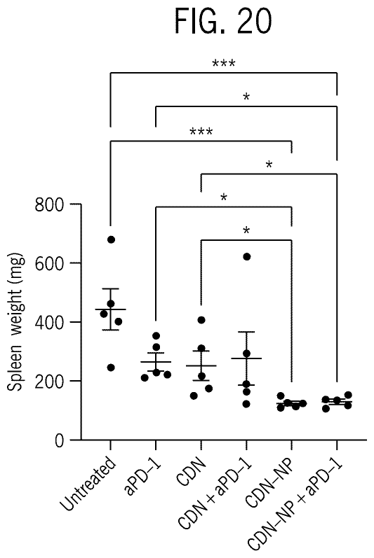

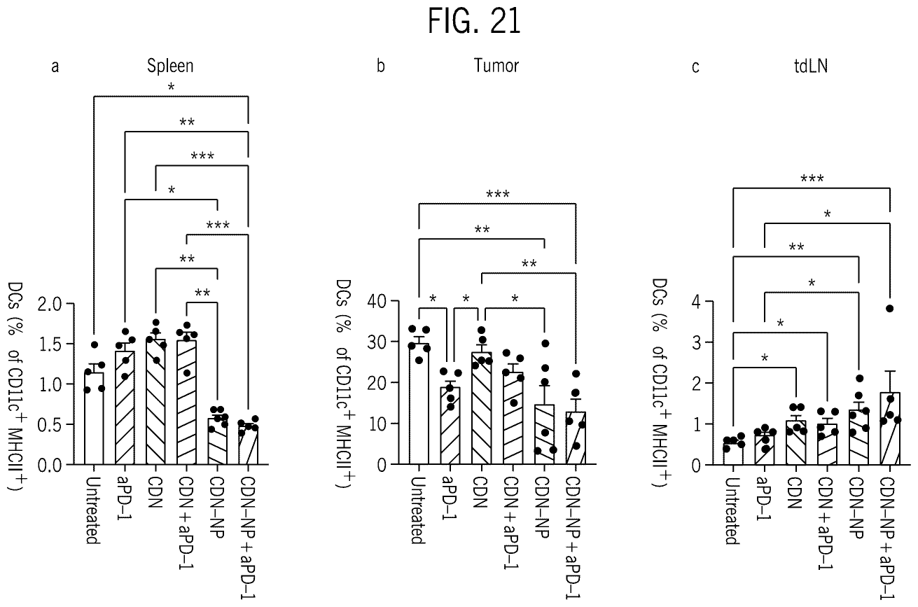

We next evaluated how a broad distribution of STING agonist molecules could lead to a wide magnitude of proinflammatory cytokines and chemokines, which are expected to cause macrophage and dendritic cell maturation, and CD8+ T cell cross-priming for adaptive tumor killing45. Here, we analyzed STING-driven cytokine production in the plasma, tdLN, TME, and spleen after i.v. administration of CDN-NPs (FIG. 11(j)). Compared with free CDN and empty NP (NP control), CDN-NP increased the expression of IFN-Is and IFN-IIs across all the tissues. Concordantly with our BD data, the highest IFNs expression was observed in the spleen (1240-fold for IFN-α, 340-fold for IFN-b, and 840-fold for IFN-g compared to untreated control). Consequently, immunosuppressive IL-10 cytokine was also upregulated across all the tissues (5-fold in TME, 28-fold in tdLN, 8-fold in spleen, and 17-fold in plasma compared to untreated control), acting as an endogenous negative regulator of STING activation. CDN-NP treatment also resulted in an elevated expression of pro-inflammatory cytokines, such as IL-17 (95-fold in TME, 103-fold in tdLN, 417-fold in spleen, and 113-fold in plasma), IL-12-p70 (7-fold in TME, 113-fold in tdLN, 29-fold in spleen, and 56-fold in plasma) and TNFa (41-fold in TME, 14-fold in tdLN, 45-fold in spleen, and 160-fold in plasma) (FIGS. 19, 11(j)). CDN-NP highly increased the expression of other cytokines involved in T-cell priming and activation such as IL-4 (109-fold in TME, 184-fold in tdLN, 910-fold in spleen, and 1387-fold in plasma) and IL-9 (6-fold in TME, 141-fold in tdLN, 382-fold in spleen, and 564-fold in plasma) cytokines. We also characterized leukocyte chemokine expression across tissues, observing a significant increase after CDN-NP treatment. CDN-NP increased the expression of IP-10 (10.5-fold), CXCL1 (4.5-fold), CCL3 (11.5-fold), and CCL2 (9.1-fold) in the TME (FIGS. 19, 11(J)), which are critical for antitumor T-cell activation and recruitment.

### CDN-NPs Induce a Potent Antitumor Response and Improve Efficacy, Compared to Free CDN, in Multiple Tumor Models

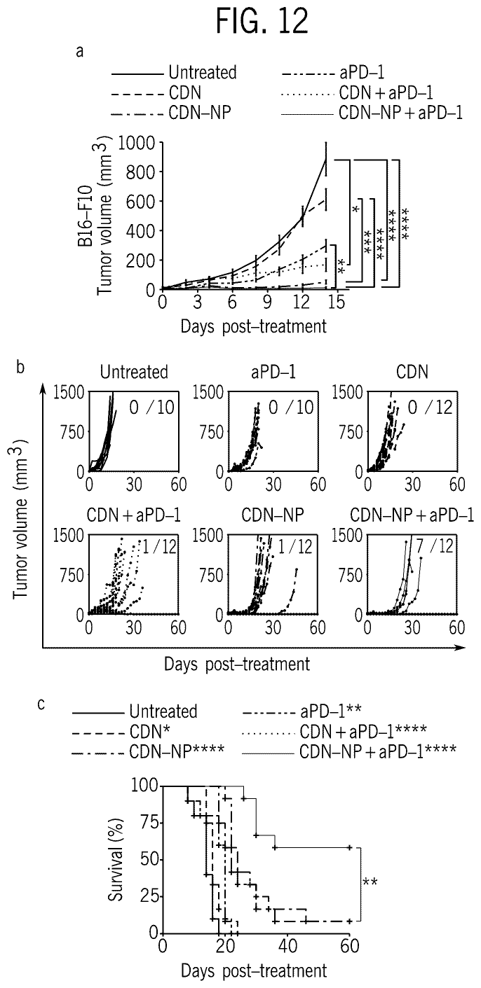

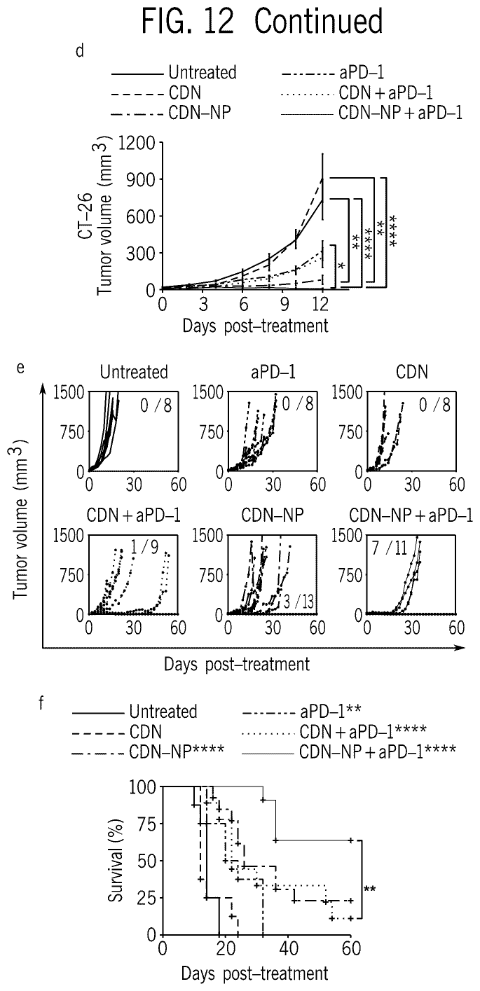

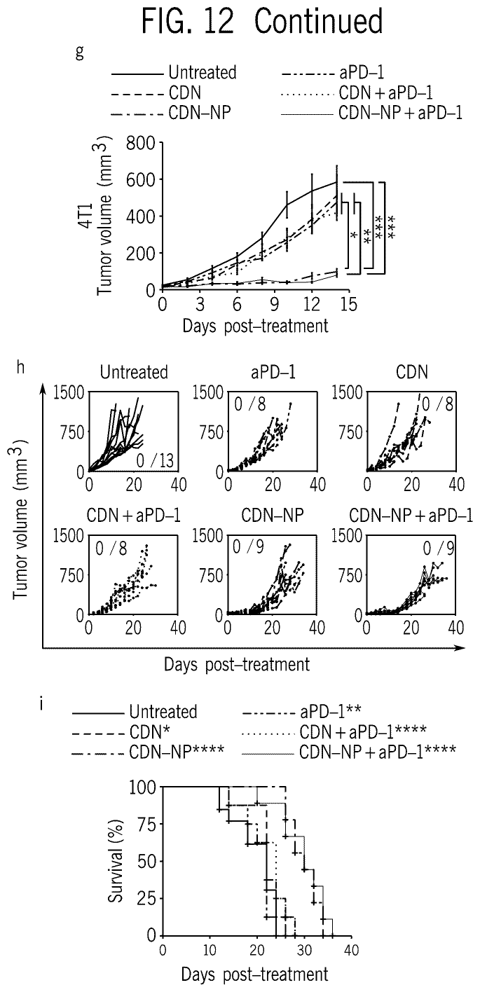

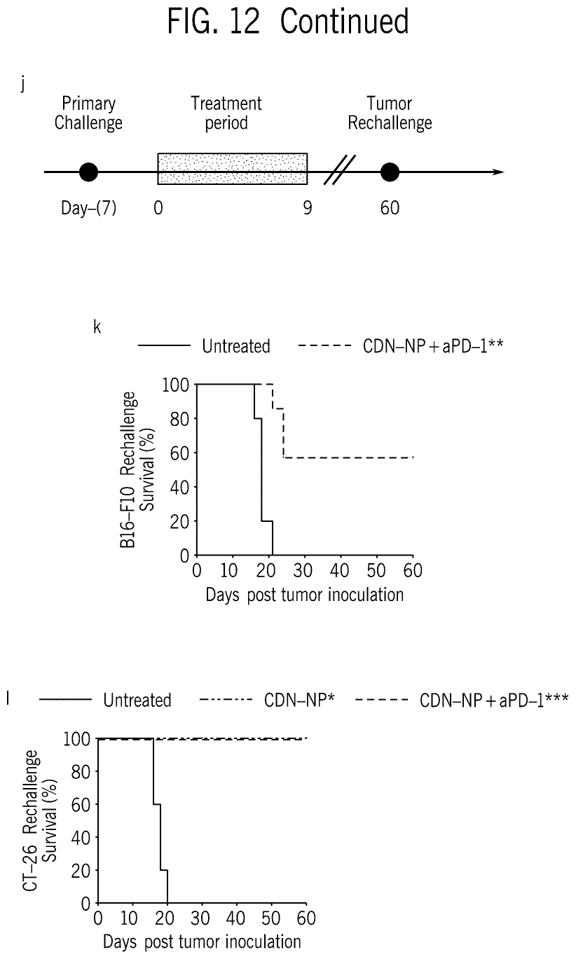

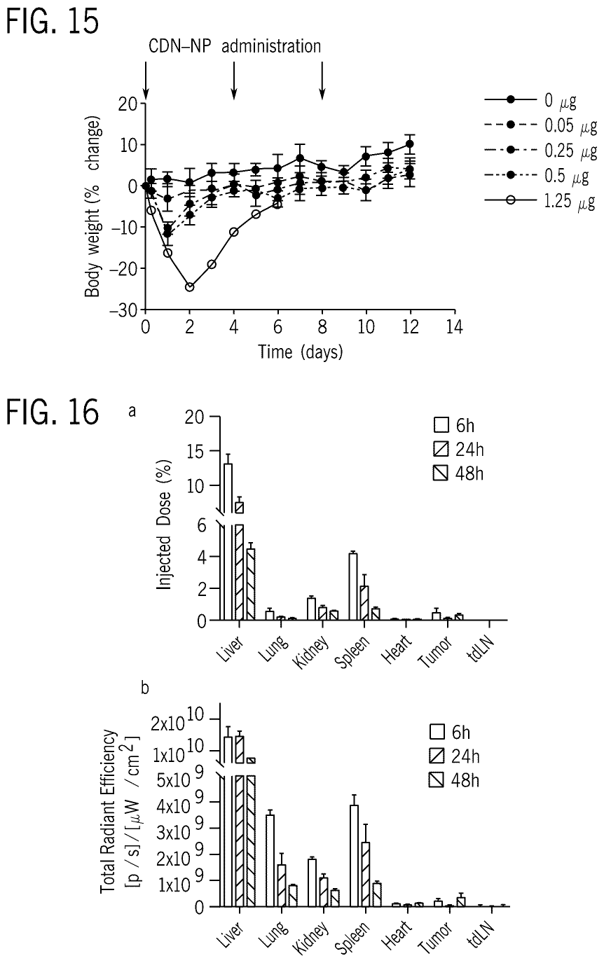

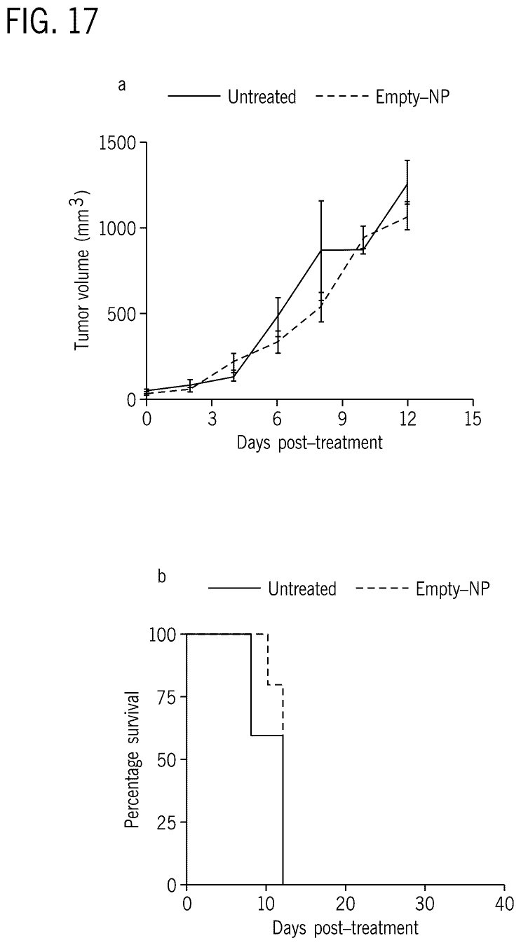

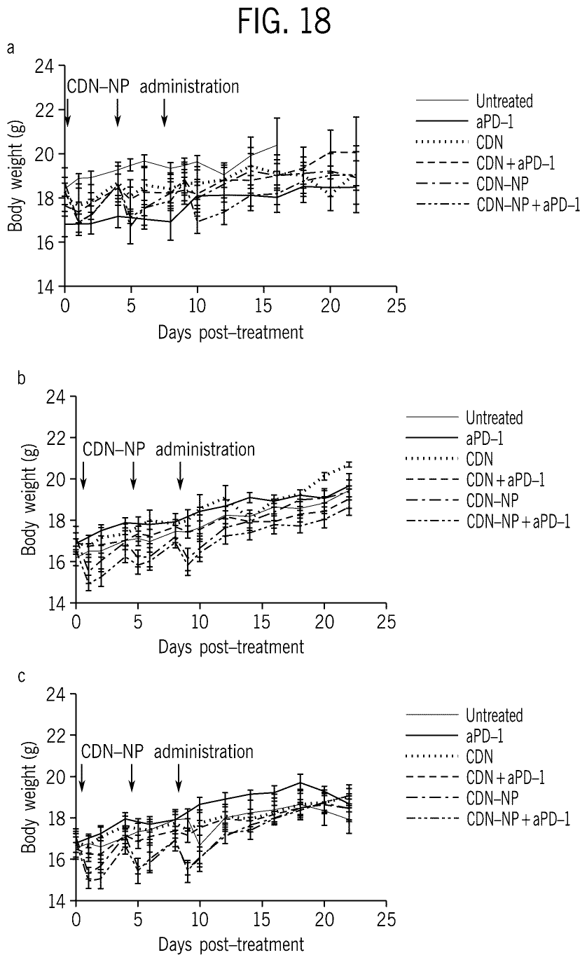

CDN conjugation to pBAE polymer increased CDN potency, where a dose as low as 1.25 μg of CDN per mouse was found to be the maximum tolerated dose (MTD) (FIG. 15). We found that 0.5 μg of CDN per mouse was well tolerated (with transient body weight loss) and it was selected for all therapeutic and functional studies. To evaluate the therapeutic efficacy of CDN-NPs in combination with Immune Checkpoint Blockade (ICB), we first utilized a poorly immunogenic murine melanoma model (B16-F10). Mice were injected intravenously with free CDN or CDN-NPs 7 days after tumor inoculation (average tumor volume ˜30-50 mm³). To evaluate the combination with ICB, aPD-1 mAb was dosed intraperitoneally 24 h after CDN therapy. This regimen was repeated for a total of three times with treatment every fourth day. Treatment with CDN-NP resulted in a significant delay in tumor growth and a corresponding increase in the survival time compared to the equivalent dose of free CDN, which resulted in only a modest suppression of tumor growth and no increase in survival (FIGS. 12(a)-12(c)). Importantly, the empty particle treatment did not affect tumor growth (FIG. 17). Slight benefit was observed when aPD-1 mAb was combined with free CDN, where a delay in the tumor growth and increased survival was observed. Interestingly, the combination of CDN-NP with anti-PD-1 markedly reduced tumor growth and 60% of the animals completely rejected tumors with no evidence of residual tumor two months post treatment (FIGS. 12(a)-12(c)). These results confirm the synergistic effect of STING agonists and ICB50, however, this synergy was greatly accentuated using CDN-NPs, resulting in tumor remission in most mice in the present study.

To evaluate how STING agonist therapy works in different tumor models we treated mice bearing CT-26 or 4T1 tumors, which are of murine colon and murine breast origin respectively. CT-26 represents a more immunogenic tumor and 4T1 is a very poorly immunogenic tumor known to be resistant to ICB. CT-26 tumors significantly responded to CDN-NP treatment, even more so than the B16-F10 tumors. Treatment with CDN-NP resulted in an inhibition in the tumor growth and an increase in the survival. In contrast to poorly immunogenic B16-F10 melanoma, almost 30% of the animals were completely cured without the presence of ICB. In addition, almost 70% of mice showed complete regression when CDN-NP was combined with aPD-1 mAb therapy (FIGS. 12(d)-12(f)), which is like the survival outcomes observed in the melanoma model to the combination therapy. A more restrained therapeutic response was observed in mice bearing 4T1 tumors (FIGS. 12(g)-12(i)), whereby treatment with CDN-NP resulted in an inhibition in the tumor growth rate and a significant increase in the survival time compared to the equivalent dose of free CDN. However, no beneficial effect was observed when free CDN or CDN-NP were combined with aPD-1 mAb. These data affirm that the unique biology and tumor immune microenvironment of different tumors can directly impact the efficacy of STING agonism-induced antitumor immune responses, and immunotherapy more broadly.

To understand if our therapeutic combination was able to generate immune memory, 60 days posttreatment we rechallenged initially cured mice with the same cell type they rejected (i.e. B16-F10 cells for mice that were cured of B16-F10 melanoma tumors, likewise for CT-26) (FIG. 12(j)). We observed that 60% (FIG. 12(k)) and 100% (FIG. 12(l)) of mice rejected B16-F10 and CT-26 tumors respectively, whereas aged-matched naive mice all rapidly succumbed to their tumors. Encouragingly, the combination of CDN-NP and aPD-1 mAb was able to generate a robust immune memory response in a majority of initially cured mice, with slightly more responsiveness in the CT-26 model, which is the most immunogenic of the ones used in this study.

### Systemic Delivery of CDN-NP Activates Innate and Adaptive Immune Responses

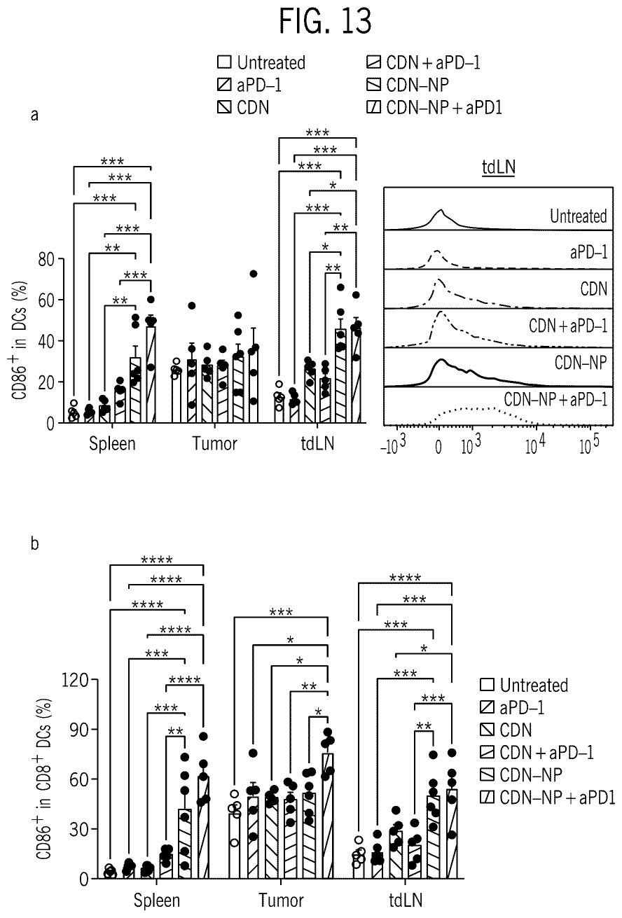

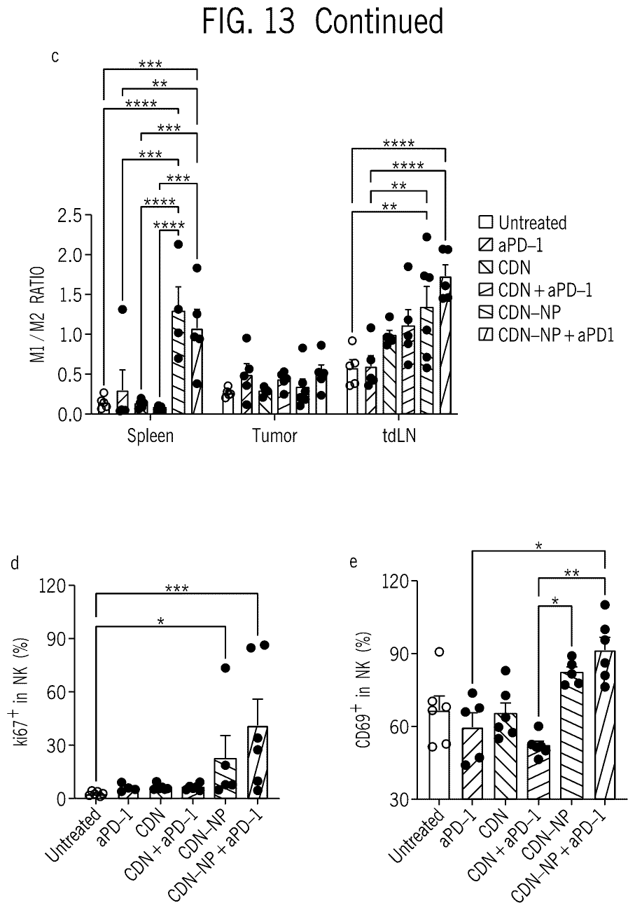

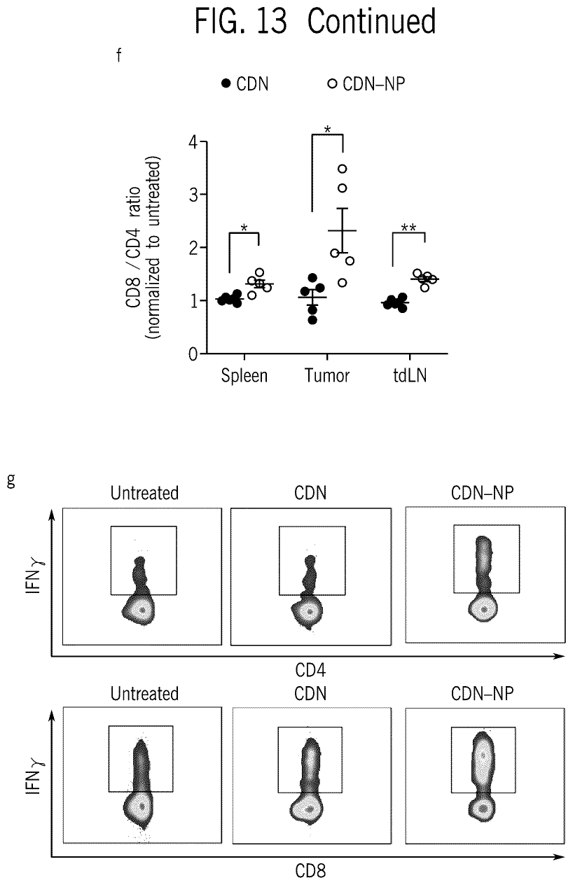

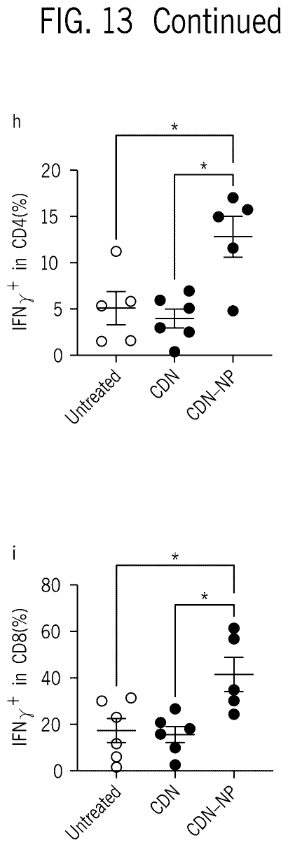

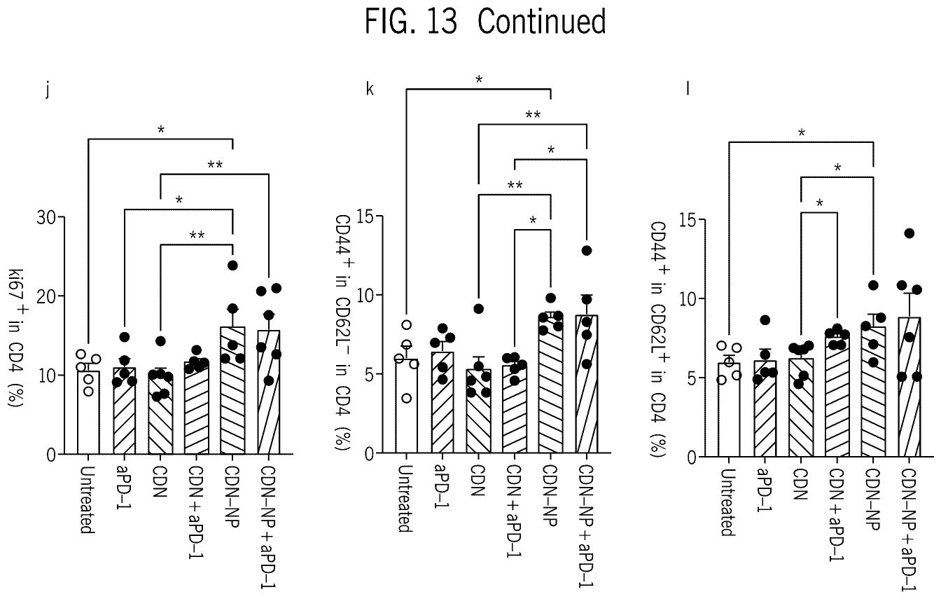

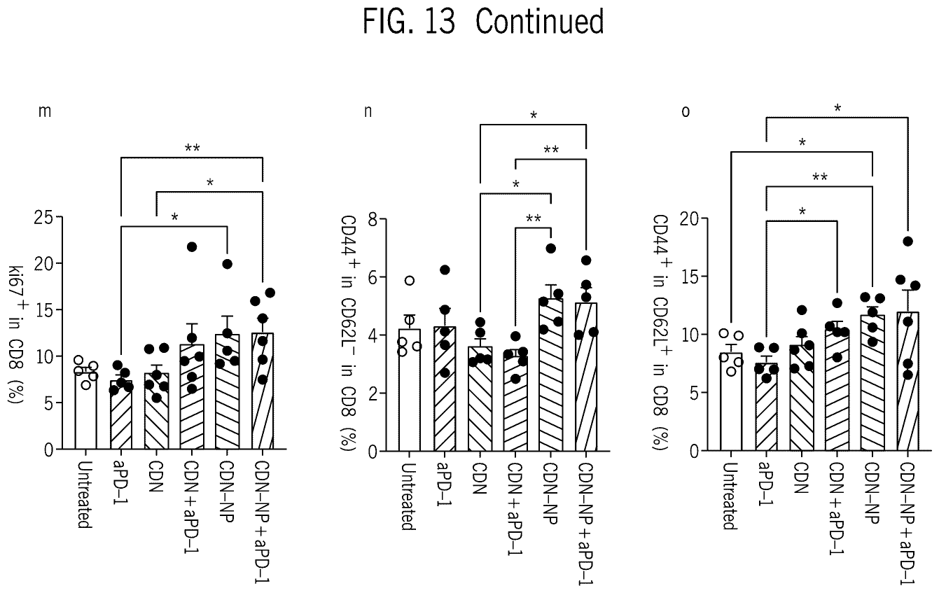

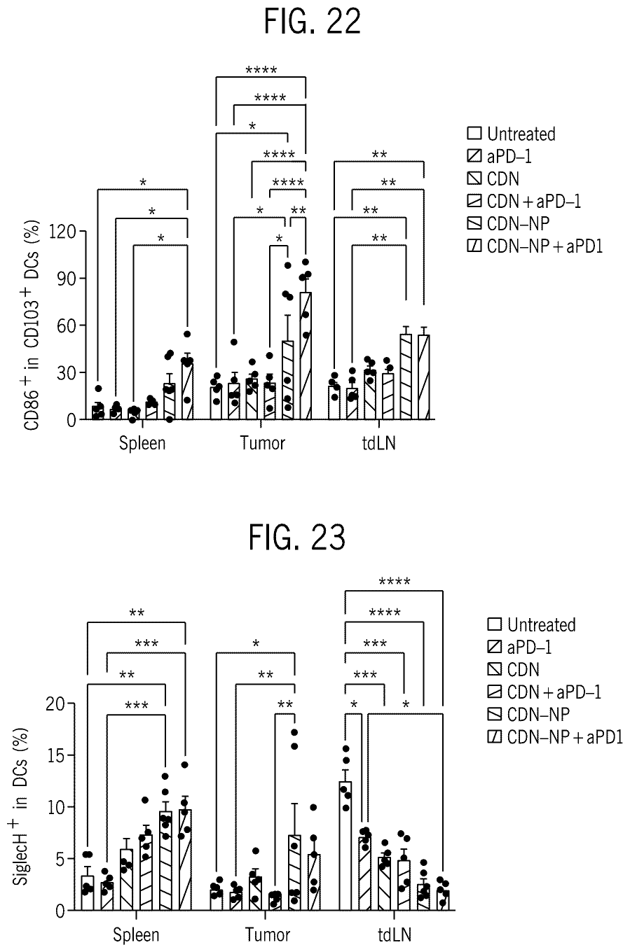

To understand the mechanism by which the CDN-NPs resulted in enhanced antitumor activity, we characterized the composition and the phenotype of the immune cell populations in the TME, tdLN, and spleen in B16-F10 melanoma tumor-bearing mice two days post CDN-NP treatment. DC expression of CD86 in the tdLN and spleen was significantly increased (FIG. 13(a)), consistent with the enhanced CDN accumulation after CDN-NP intravenous administration (FIGS. 11(d), 11(e)). The combination of ICB with CDN therapies further increased CD86 expression on DCs compared to CDN monotherapy in the spleen (47.1±5.4% CDN-NP+aPD-1 vs. 31.6±5.7% CDN-NP alone, and 15.7%+1.8 free CDN+aPD-1 vs. 8.4±1.3% free CDN alone group). A similar trend was observed in the tdLN. Interestingly, the combination of CDN-NP with aPD-1 mAb significantly activated migratory (CD103⁺) and tissue resident (CD8⁺), cross-presenting cDC1 subsets from the TME, tdLN and spleen (FIGS. 22 and 13, respectively). The percentage of plasmacytoid DCs (pDCs) was increased in the spleen and TME after CDN-NP treatment (FIG. 23)—producing high levels of type I interferons (IFN-I) in response to CDN therapy. In contrast, the overall percentage of pDCs in the tdLN was decreased due to the influx of DCs after CDN-NP treatment.

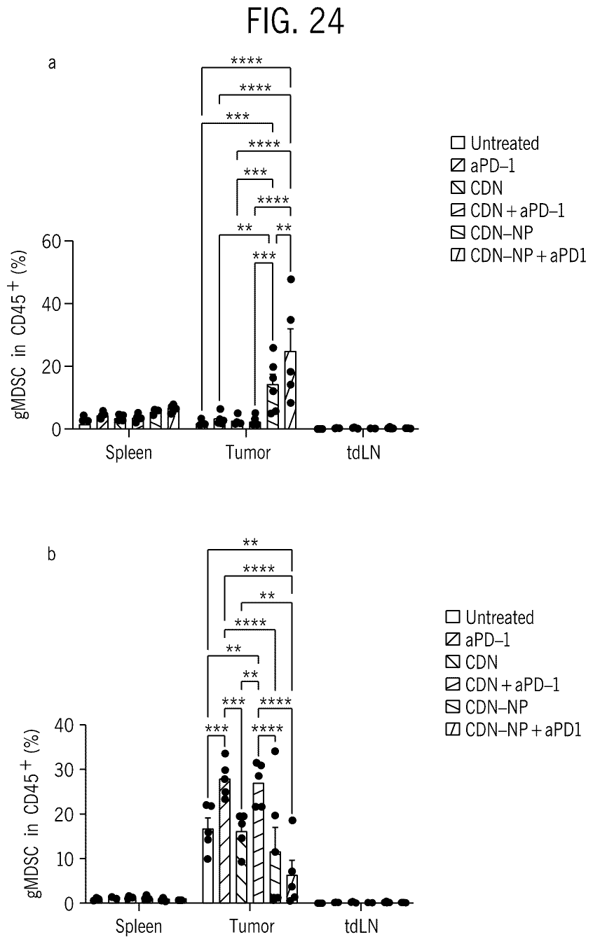

Furthermore, in the spleen and tdLN, we observed a shift in immunosuppressive macrophages (CD206^{hi}) to a more M1-like state (CD86⁺) in mice treated with CDN-NPs alone or with ICB (FIG. 13(c)), suggesting repolarization of anti-inflammatory M2-like macrophages to a more immunostimulatory phenotype or enhanced recruitment of immunostimulatory, M1-like, macrophages. CDN-NPs promoted an influx of immunosuppressive granulocytic myeloid derived suppressor cells (gMDSC) in the TME and spleen (FIG. 24(a)), a phenomenon that has previously been reported and is thought to be due to a regulatory negative feedback mechanism in response to STING-mediated inflammation. Consequently, percentage of monocytic MDSC (mMDSC) observed by flow cytometry was reduced in those tissues (FIG. 24(b)). CDN-NPs also increased proliferation (Ki67⁺) and activation of NK cells in the TME (FIGS. 13(d), 13(e)), which was encouraging given the role of NK cells in STING-based immune responses.

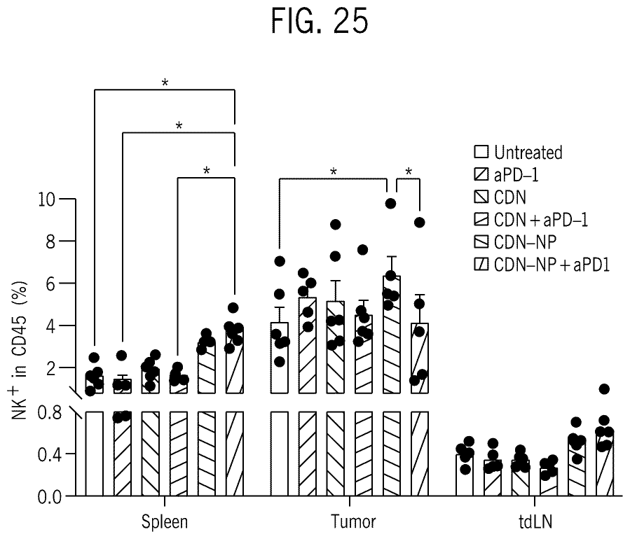

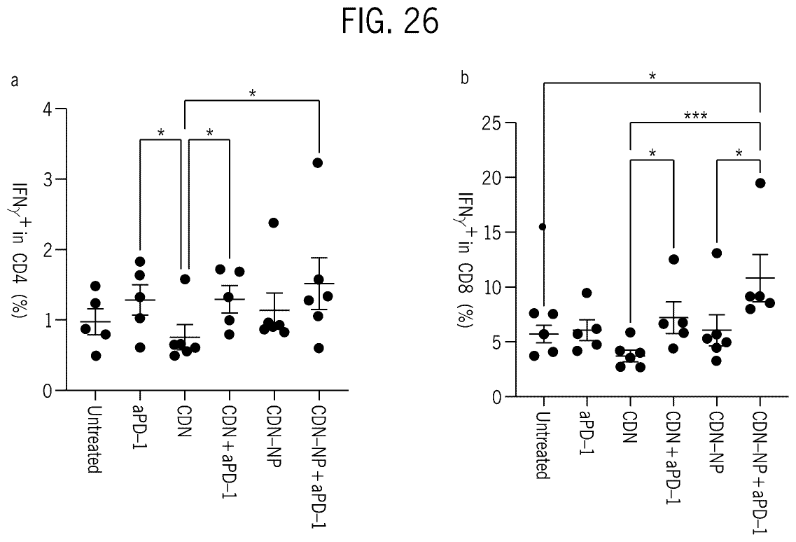

Analysis of tumoral T-cell infiltrates revealed a 2-fold increase in the CD8/CD4 T-cell ratio (a common prognostic biomarker of responsiveness to immunotherapy) in the TME seven days post CDN-NP treatment compared to equal doses of free CDN (FIG. 13(f)) with a similar trend observed in the tdLN. Upon ex vivo restimulation we observed that the proportion of CD8⁺ T cells secreting IFN-γ in the TME were significantly increased in mice that were treated with CDN-NP compared to the equivalent dose of free CDN or untreated mice (41.5±7.4% for CDN-NP vs.17.4%±5.2 and 15.7±3.5% in untreated and free CDN groups, respectively) (FIGS. 13(g), 13(i)). This was similarly observed in CD4⁺ T cells (12.8±2.2% in the CDN-NP group versus 5.1±1.8% and 4.0±1.0% in untreated and free CDN groups, respectively) (FIG. 13(h)). In line with their cytotoxic effector function, a higher percentage of IFN-γ expressing cells were CD8^{±} relative to CD4, which was also observed in the tdLN (FIG. 26).

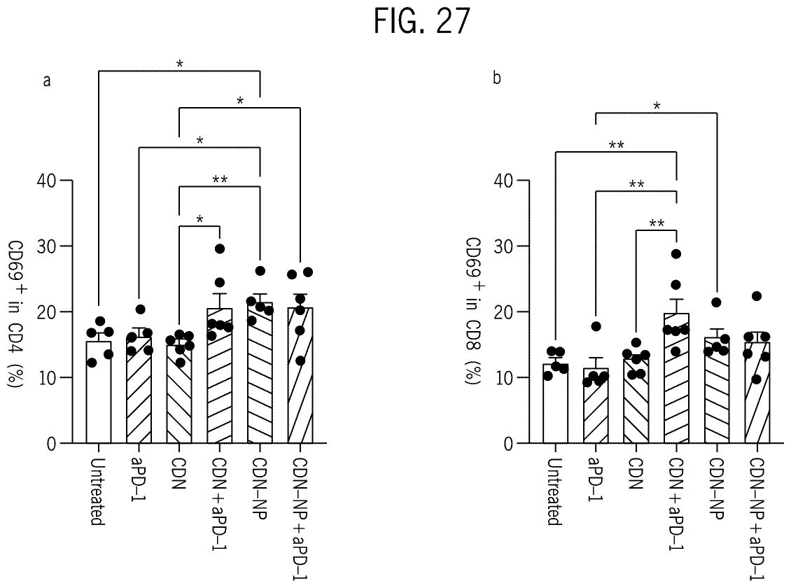

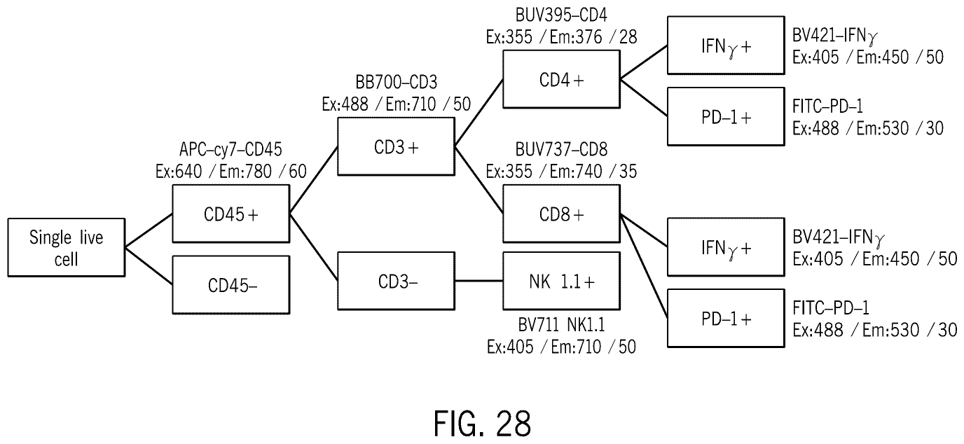

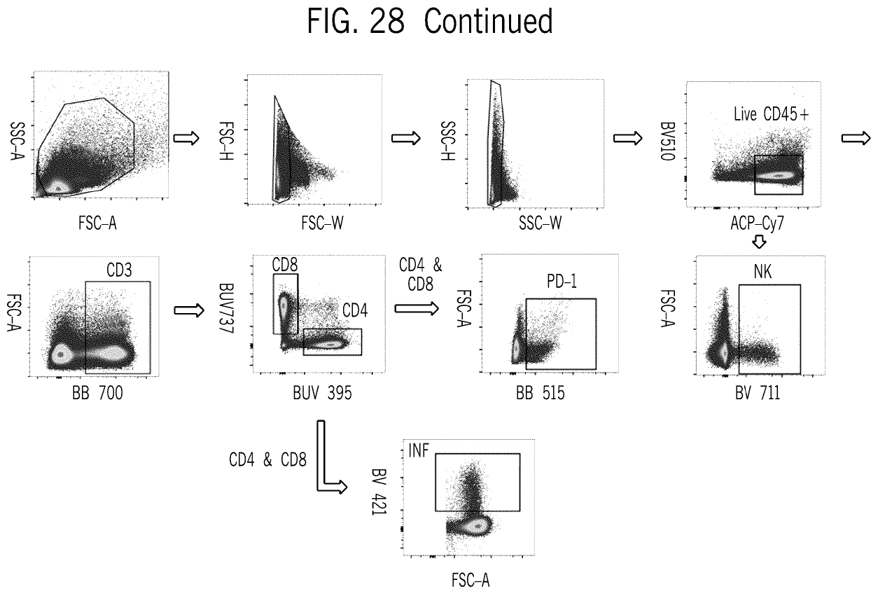

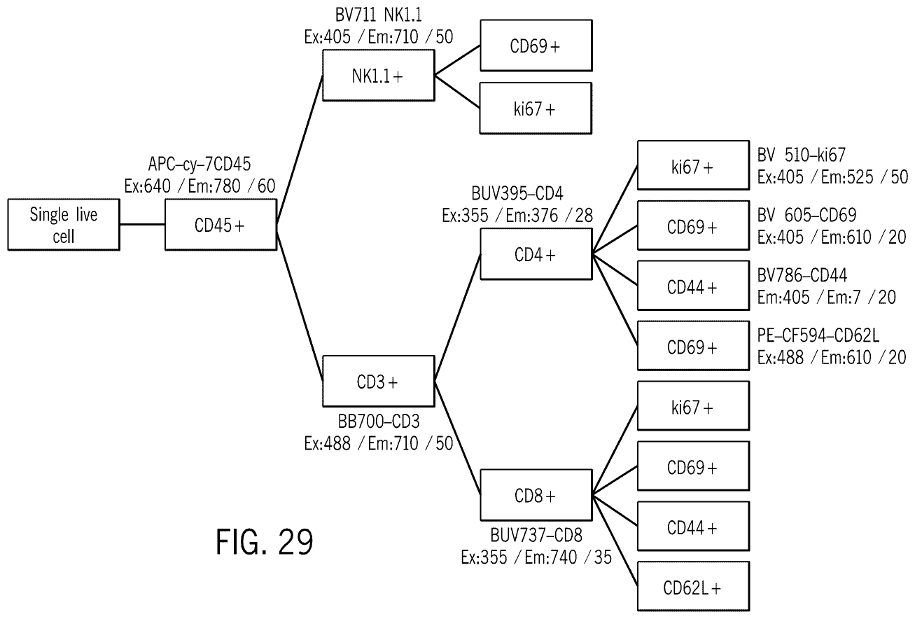

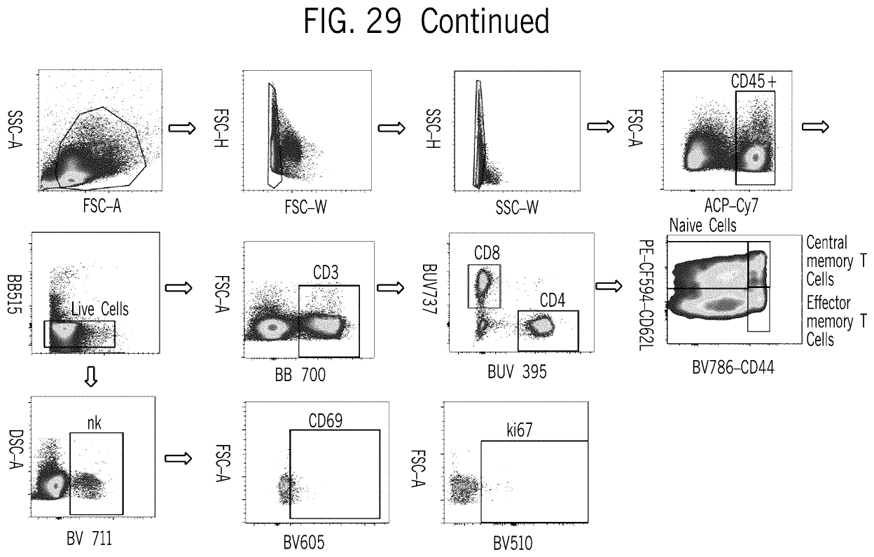

Given that a majority of tumor bearing mice were cured and rejected a second challenge with tumor cells when treated with CDN-NP and ICB but no other treatment types, we probed the immune cell landscape for the cell populations responsible for the induction of immunological memory. Seven days after a single treatment, the TME and tdLN were collected and analyzed using flow cytometry to examine memory immune cells. We first observed an increase in CD4 and CD8 T cell proliferation (ki67⁺) and activation (CD69⁺) in the tdLN after CDN-NP treatment compared to untreated or free CDN groups (FIGS. 13(j)-13(m), 27). Interestingly, the percentages of CD8⁺CD44^{hi}CD62L^{h} central memory T cells (TCM) and CD8+CD44hiCD62Llo effector memory T cells (TEM) were both increased in CDN-NP treated mice (FIGS. 13(n), 13(o)). A similar profile was also observed in CD4+ cells, where CDN-NP treated mice with aPD-1 mAb showed higher percentages of TCM (9.6±1.5%) and TEM (8.8±1.82%) cells compared with untreated (5.9±0.4% and 6.0±0.8%, respectively) and free CDN (6.2±0.4% and 5.3±0.8%, respectively) groups (FIGS. 13(k), 13(1)). These results provide evidence that treatment with CDN-NPs promotes the generation of antitumor immune and subsequent memory cell formation, which is not present in any other treatment group.

Interestingly, immune memory formation after intratumoral administration of the STING agonist Aduro S100 was found to be dependent on the dose and therefore magnitude of the response, as high doses resulted in an ablative TNF-α driven primary tumor clearance but failed to generate immune memory, and a lower, immunogenic dose was more efficient when combined with ICB 50. It is possible that, although delivered systemically, the dose used in this study was within the immunogenic range and explains why such a significant benefit was achieved with ICB.

### Cancer-Cell STING Activation is Inconsequential to Promoting Anti-Tumor Immunity, However, Cancer Cells can Act as a Reservoir for CDN-NPs

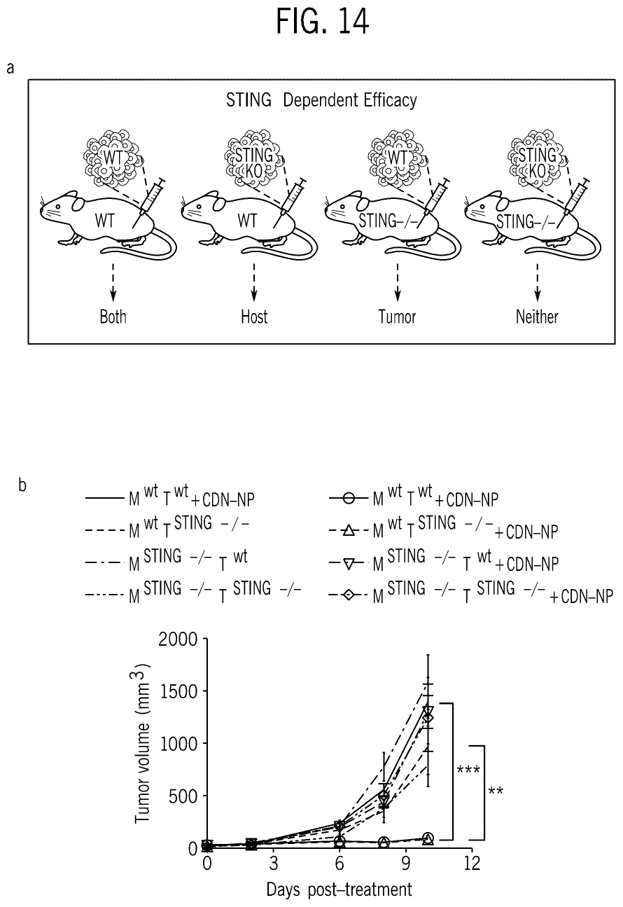

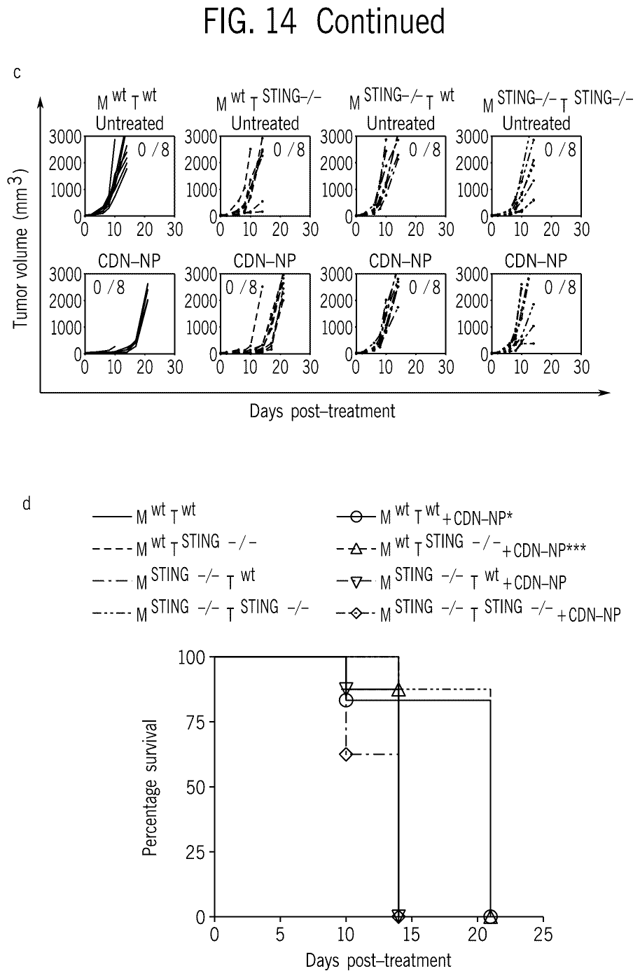

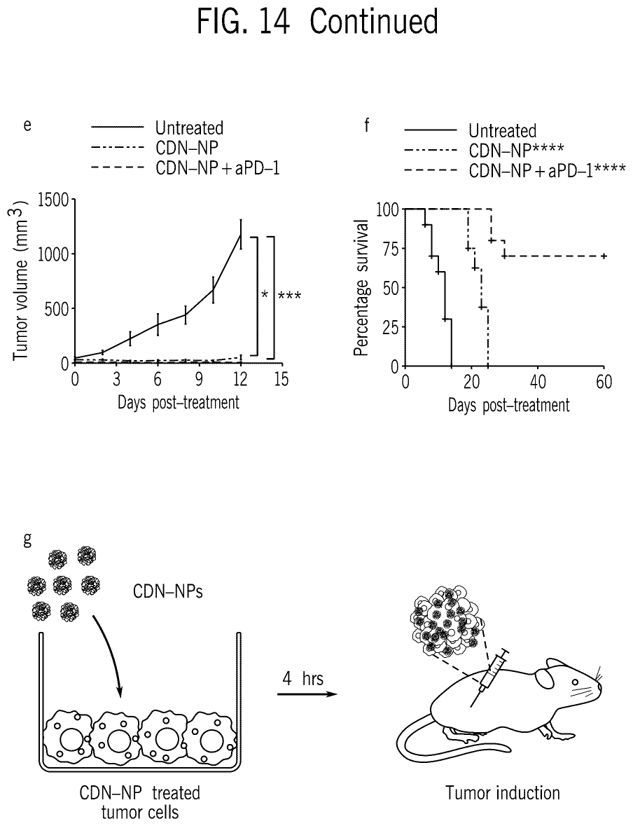

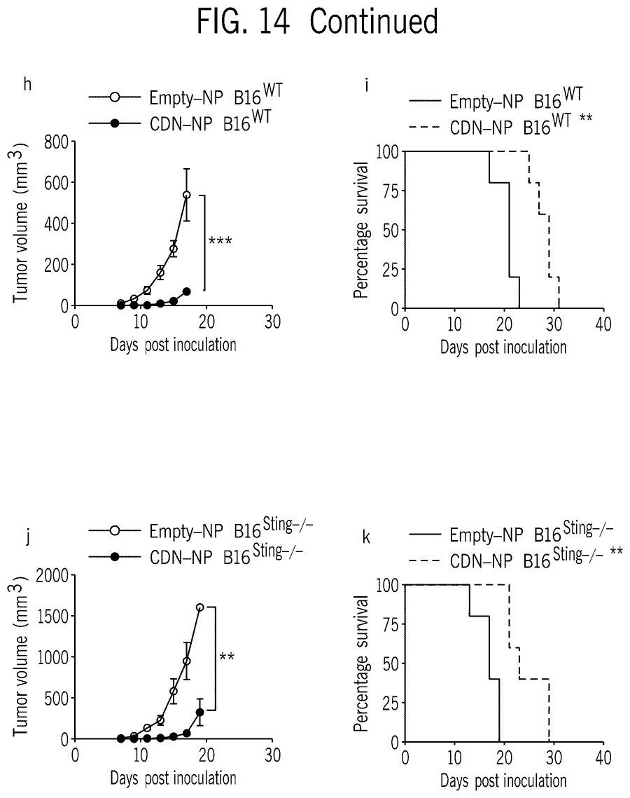

Immune cell analysis suggested that CDN-NP activated innate immune cells and T cells in the TME, the tdLN, and the spleen. To further investigate the contribution of the host immune system to the robust therapeutic efficacy of CDN-NPs, STING KO (M^{STING-/-}) or wild type (M^{wt}) mice were inoculated with either B16-F10 WT (T^{wt}) or STING KO B16-F10 (T^{STING-/-}) tumor cells (FIG. 14(a)). Consistent with previous efficacy studies, the treatment was initiated 7 days post-tumor induction (tumor volume=30-50 mm³) and animals were dosed 3 times, every four days, over the course of 8 days. In agreement with our previous results, treatment with CDN-NP in M^{wt}T^{wt} mice resulted in an inhibition in tumor growth (FIGS. 14(b), 14(c)) and a significant increase in overall survival (FIG. 14(d)) compared to the corresponding untreated mice. Similar results were observed when M^{wt}T^{STING-/-} mice were treated with CDN-NP, whereas the therapeutic efficacy of CDN-NP in B16^{wt} or B16^{STING-/-} tumor-bearing M^{STING-/-} mice was completely abolished. These results confirm that CDN-NP elicits tumor regression through activation of host STING and that tumor cell STING is not sufficient or required to promote anti-tumor immunity. Once we confirmed host STING is required for CDN-NP antitumor immunity and that efficacy of CDN-NPs is STING-specific, we turned our attention to understanding the role of the spleen in CDN-NP antitumor efficacy, given the robust splenic accumulation of CDNNPs and activation of splenic immune cell populations. To do this, we induced B16-F10 tumors in splenectomized mice (SpleenX C57BL/6 mice from Charles River Laboratories) and treated the mice 7 days post tumor inoculation with intravenous injection of CDN-NPs alone or with ICB, three times every four days. To our surprise, we found that the absence of a spleen did not have any bearing on the ability of mice to reject their primary tumors when treated with CDN-NP alone and in combination with ICB, with 70% of mice cured of their primary tumor in the combination group (FIGS. 14(e), 14(f)). Finally, we were interested to study the role of cancer cells after CDN-NP delivery. As we previously observed, cancer cells efficiently internalize CDN-NPs and they can act as a CDN-NPs reservoir (FIGS. 10(k)-10(n)). Here, we studied whether CDN-NPs from melanoma cancer cells can be transferred to other cell lines in vivo. Wild type B16-F10 (B16^{wt}) and STING KO B16-F10 (B16^{-/-}) cancer cells were incubated with CDN-NP or empty-NP 4 h prior to tumor inoculation (FIG. 14(g)). CDN-NP treated B16 wt cells resulted in a delay in the tumor growth and survival compared to Empty-NP treated cells in wild type C57BL/6 mice (FIGS. 14(h)-14(i)). Similar results were obtained with CDN-NP treated B16^{-/-} cells (FIGS. 14(j), 14(k)), confirming that tumor cell STING is not driving tumor growth inhibition and suggesting that CDN-NP may be released from the cancer cells and consequently internalized by the host immune cells, resulting in STING activation and the downstream immune anti-tumor effect.

Through considered design of drug and delivery vehicle, we have created various embodiments of a STING agonist formulation with greatly enhanced potency, compared to previous formulations. A novel CDN-based STING agonist conjugated to pBAE NPs, CDN-NPs, was found to induce marked tumor regression after intravenous administration in a STING-specific manner at sub-microgram doses. Systemic delivery of CDN-NPs induced phenotypic changes in different myeloid populations and increased expression of proinflammatory markers by DCs and macrophages in the TME, tdLN, spleen, and liver, likely to be responsible of the observed antitumor immune response. When combined with ICB, in melanoma and colon cancer models, complete and durable antitumor responses with immunological memory were generated, demarcated by a dramatic shift in the TME from immunologically quiescent to stimulatory. Mechanistically, CDN-NPs are delivered to immune cell populations whereby, upon endosomal escape, self-immolative cleavage of the CDN from the NP leads to pronounced STING activation and downstream antitumor responses. We observed that cancer cells act as a CDN-NPs reservoir, enabling transfer to proximal immune cells, in vitro and in vivo. Our data encourage the understanding of the interactions between nanomaterials and biological systems in the context of STING agonist, to further improve its performance, particularly in the context of clinically-approved regimens in immune-oncology.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus ≥10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A nanoparticle composition comprising:
a first polymer conjugated to a drug by a linker to form a first polymer compound,
the first polymer compound having a net negative charge; and
a second polymer conjugated to at least one positively charged group to form a second polymer compound,
the second polymer compound having a net positive charge, and
the first polymer compound and the second polymer compound interacting electrostatically to form a nanoparticle.

2. The nanoparticle of claim 1, wherein the first polymer comprises poly(β-amino ester).

3. The nanoparticle of claim 1, wherein the second polymer comprises poly(β-amino ester).

4. The nanoparticle of claim 1, wherein the drug comprises a nucleotide.

5-7. (canceled)

8. The nanoparticle of claim 1, wherein the linker comprises an enzyme-sensitive linker.

9. The nanoparticle of claim 8, wherein the enzyme-sensitive linker comprises a cleavable linker.

10. (canceled)

11. The nanoparticle of claim 1, wherein the linker comprises one or more terminal maleimide groups.

12. (canceled)

13. The nanoparticle of claim 1, wherein the at least one positively-charged group comprises a positively-charged amino acid.

14-17. (canceled)

18. The nanoparticle of claim 1, wherein the first polymer compound comprises a drug conjugated by an enzyme-sensitive linker at each of a first end of the first polymer and a second end of the first polymer.

19. The nanoparticle of claim 1, wherein the second polymer compound comprises at least one positively-charged group conjugated at each of a first end of the second polymer and a second end of the second polymer.

20. (canceled)

21. A pharmaceutical composition comprising an effective amount of the nanoparticle according claim 1 and a pharmaceutically acceptable excipient, carrier, or diluent.

22. A method of treatment of a subject in need of a treatment for cancer, the method comprising administering an effective amount of the nanoparticle of claim 1.

23. (canceled)

24. The method of claim 22, wherein the nanoparticle contacts a first cell.

25. (canceled)

26. The method of claim 24, wherein the nanoparticle resides in the first cell for at least 24 hours.

27. The method of claim 24, wherein the nanoparticle transfers from the first cell to a second cell.

28-30. (canceled)

31. A nanoparticle composition comprising:
a polymer conjugated to a drug by a linker to form a polymer compound, the polymer further conjugated to at least one positively charged group such that
the polymer compound having a net positive charge, and the polymer compound interacting electrostatically to form a nanoparticle.

32. The nanoparticle of claim 31, wherein the polymer comprises poly(β-amino ester).

33. The nanoparticle of claim 31, wherein the drug comprises a nucleotide.

34-35. (canceled)

36. The nanoparticle of claim 31, wherein the linker comprises an enzyme-sensitive linker.

37-39. (canceled)

40. The nanoparticle of claim 31, wherein the at least one positively-charged group comprises a positively-charged amino acid.

41-48. (canceled)
